(19) United States
(12) Patent Application Publication
(10) Pub. No.: US 2023/0211011 A1
(43) Pub. Date: Jul. 6, 2023

(54) TARGETED CRYSTALLIZATION OF MIXED-CHARGE NANOPARTICLES IN LYSOSOMES FOR INDUCING SELECTIVE DEATH OF CANCER CELLS

(71) Applicants: INSTITUTE FOR BASIC SCIENCE, Daejeon (KR); UNIST(ULSAN NATIONAL INSTITUTE OF SCIENCE AND TECHNOLOGY), Ulju-gun (KR)

(72) Inventors: Bartosz Grzybowski, Ulju-gun (KR); Kristiana Kandere-Grybowska, Ulju-gun (KR); Magdalena Borkowaska, Ulju-gun (KR); Marta Siek, Ulju-gun (KR); Diana Kolygina, Ulju-gun (KR)

(21) Appl. No.: 17/800,990

(22) PCT Filed: Feb. 22, 2021
(86) PCT No.: PCT/KR2021/002208

§ 371 Date: Aug. 19, 2022

(30) Foreign Application Priority Data
Feb. 21, 2020 (KR) 10-2020-0021515

(51) Int. Cl. A61K 47/69; A61K 33/242; A61K 33/26; A61K 33/00; A61K 47/54; A61P 35/00

(52) U.S. Cl. CPC A61K 47/6929; A61K 33/242; A61K 33/26; A61K 33/00; A61K 47/54; A61K 47/548; A61K 47/542; A61P 35/00

(57) ABSTRACT

The present invention relates to a mixed-charge nanoparticle for inducing selective death of cancer cells and a use thereof. The mixed-charge nanoparticle of the present invention is localized and crystallized specifically in cancer cell lysosomes through a pH-dependent aggregation behavior due to the balance between positively charged ligands and negatively charged ligands on the surface thereof and can induce lysosomal membrane permeabilization (LMP) and lysosomal cell death mediated thereby, like cationic amphiphilic drugs (CADs) Exhibiting a cancer cell-specific death effect, the nanoparticles of the present invention can surmount the limited medical use of conventional cationic nanoparticles due to the non-specific cytotoxicity thereof. Particularly, the nanoparticles of the present invention do not exhibit toxicity to the human body and normal cells, thus finding useful applications in medical and medicinal uses such as for prevention and treatment of solid cancer, blood cancer, and tumors.

### TECHNICAL FIELD

The present invention relates to mixed-charge nanoparticles for inducing selective death of cancer cells and the use thereof, and more particularly, to mixed-charge nanoparticles comprising a positively charged ligand and a negatively charged ligand and having a positive net surface charge, the lysosomal membrane selectivity-mediated cancer cell-specific cell death effect thereof, and the use thereof for preventing or treating cancer.

### BACKGROUND ART

Most biological macromolecules are charged, and intramolecular or intermolecular electrostatic interactions due to these electrical properties correspond to key biological mechanisms (Science 1995, 268, 1144-1149; Future Med. Chem. 2010, 2, 647-666; Phys. Biol. 2011, 8, 035001). For example, nucleic acid backbones bear negative charges, protein surfaces display a patchwork of positively or negatively charged amino acid residues, and lipid bilayers presenting anionic and zwitterionic head groups enclose cells or subcellular compartments. Efforts have been made to develop charged therapeutics that target charged entities by using these electro-biological properties (Angew. Chem. Int. Ed. 1998, 37, 2755-2794). In particular, various literature has reported that nanoparticles (NPs) decorated with charged polymers (Small 2014, 10, 4230-4242; Med. Chem. 2015, 15, 1179-1195) or charged ligands (Small 2014, 10, 4230-4242; J. Am. Chem. Soc. 2012, 134, 6920-6923; Nat. Nanotechnol. 2009, 4, 457-463) can be used as tools to control the function of cells.

However, it has been reported that negatively charged nanoparticles (e.g., nanoparticles covered with carboxylate-terminated or thiolated oligonucleotides) are poorly uptaken from serum-containing media and into adherent mammalian cells other than phagocytes (macrophages and dendritic cells) (Int. J. Nanomedicine 2014, 9 Suppl 1, 51-63, Bioconjug. Chem. 2010, 21, 2250-2256, Nat. Mater. 2008, 7, 588-595, Int. J. Nanomedicine 2012, 7, 5577-5591).

On the other hand, it has been reported that positively charged nanoparticles can be depolarized by interaction with the cell surface membrane, and are efficiently internalized into the cell, but they can easily escape endosomes through cellular membranes generating transient nanopores, and as a result, are highly toxic (ACS Nano 2008, 2, 85-96; ACS Nano 2010, 4, 5421-5429). To overcome this limitation, nanoparticles covered with charged monolayers diluted with neutral/hydrophobic ligands were also designed, but such “charge-diluted” cationic NPs are not necessarily less cytotoxic (both reduced cytotoxicity (ACS Nano 2010, 4, 5421-5429) and enhanced cytotoxicity (Nat. Nanotechnol. 2020, 15, 331-341) have been observed). On the other hand, “striped” anionic NPs with alternating anionic-hydrophobic ligand domains are membrane-permeant, which is desirable only for selected applications.

Nanoparticles covered with zwitterionic, charge-balanced ligands (or mixtures of ligands) (Small 2014, 10, 4230-4242; ACS Nano 2013, 7, 6244-6257) were shown to resist aggregation and nonspecific adsorption of serum proteins enabling efficient renal clearance in vivo (ACS Nano 2015, 9, 9986-9993). For zwitterionic NPs functionalized with a mixture of ligands, aggregation in acidic pH of tumors and enhanced uptake of such aggregates was demonstrated (Small 2014, 10, 4230-4242; ACS Nano 2013, 7, 6244-6257), but more often zwitterionic NPs are not readily internalized by cells and thus cannot be used to target intracellular compartments.

Due to the above-described electrical properties of nanoparticles, electrical properties in a cell's complex microenvironment can be changed by local ionic strength and pH, etc. depending on the location in the cell, and it is hard to ensure that charged nanoparticles navigate through a cell's complex microenvironment without undergoing undesired aggregation or without binding to some decoy structures.

Meanwhile, lysosomal organelles comprise a dynamic system of acidic vesicular compartments receiving cargoes from the plasma membrane via endocytosis or from the cytoplasm through autophagy, all ultimately destined for degradation and/or recycling. In cancer cells, these degradation pathways are deregulated, causing various alterations in the structure and function of lysosomal membranes and ultimately rendering these cells more susceptible to lysosomal membrane permeabilization (LMP) by various endogenous (p53 activation, oxidative stress) and exogenous (cationic amphiphilic drugs, CADs) triggers (Wiley (2016); Acta 1793, 746-754 (2009)). Lysosomal cell death (LCD) triggered by LMP commonly bypasses the classical caspase-dependent apoptosis pathway, opening up a new strategy for targeting apoptosis- and drug-resistant cancers (Int. J. Mol. Sci. 19, 2256 (2018); Cell Cycle 9, 2305-2309 (2010)). A handful of small-molecule compounds (antimalarial (Int. J. Mol. Sci. 19, 2256 (2018)), antihistamine (Cancer Cell 24, 379-393 (2013)) and anticancer (Nat. Rev. Drug Discov. 1, 491-492 (2002)) drugs, as well as leads from LMP screening assays (for example, thioridazine, fluphenazine or toremifene) are under clinical trials (Clinicaltrials.gov, accessed 2018-11-17) and are also known to cause LMP (Assay Drug Dev. Techn. 14, 489-510 (2016)). None thereof, however, were specifically designed to target the LCD pathway (Assay Drug Dev. Techn. 14, 489-510 (2016)); most have low cancer selectivity (Int. J. Mol. Sci. 19, 2256 (2018)) and their effects are not always lysosome-specific. Therefore, the development of drugs capable of inducing cancer cell-specific LSD by LMP has strong potential as new therapeutic agents for various anticancer therapies, cancer cell killing therapies, and drug-resistant cancers.

Under this technical background, the present inventors have been studying charged nanoparticles, both in the context of electrostatic self-assembly and for their uses in nanobiomedicine (Science 312, 420-424 (2006); Nano Lett. 7, 1018-1021 (2007); Small 5, 1600-1630 (2009); Nanoscale 8, 157-161 (2016); ACS Nano 10, 5536-5542 (2016); Angew. Chem. Int. Ed. 55, 8610-8614 (2016)). In order to overcome difficulties in intracellular internalization of anionic nanoparticles and the non-specific cytotoxicity of cationic nanoparticles, nanoparticles covered with positively and negatively charged ligands (mixed charge nanoparticles, [+/−] NPs) have been developed. As a result of studying the properties of the mixed-charge nanoparticles by controlling the ratio of cationic/anionic ligands, it was confirmed that the mixed-charge nanoparticles could selectively kill gram-positive or gram-negative bacteria, were maintained in mice for a long time (Angew. Chem. Int. Ed. 55, 8610-8614 (2016)), and could be precipitated and crystallized in vitro in a pH-dependent manner (J. Am. Chem. Soc. 135, 6392-6395 (2013); J. Phys. Chem. C 120, 4139-4144 (2016)).

Based on the pH-dependent in vitro precipitation and crystallization properties of the mixed-charge nanoparticles, the present inventors have made extensive efforts to develop mixed-charge nanoparticles that can be internalized specifically into cancer cells, can be localized in a target compartment in vivo, and can induce lysosome-dependent cell death (LSD). As a result, the present inventors have produced novel mixed-charged gold nanoparticles having a positive net surface charge by decorating the surface of nanoparticles with a mixture of positively and negatively charged ligands, and have found that the produced nanoparticles can be clustered on the cell surface, may enter the cell, may accumulate in multivesicular endosomes, may be transported to the lysosome, and may be aggregated by the specific pH environment inside the lysosome, causing progressive damage to the lysosomal membrane, resulting in cell death. In particular, the present inventors have found that, in cancer cells, the mixed-charge nanoparticles of the present invention are not released out of the lysosome after aggregation, and thus induce cell death, whereas in normal cells, the nanoparticles may be rapidly released out of the cell, and thus induce cancer cell-specific death, and this cancer cell-specific death characteristic is due to the size of the nanoparticles, the charge balance resulting from the ratio of positively charged ligands to negatively charged ligands, and the surface charge, thereby completing the present invention.

The above information disclosed in this Background section is only for enhancement of understanding of the background of the present invention. Therefore, it may not contain information that forms conventional art that is already known in the art to which the present invention pertains.

### SUMMARY OF THE INVENTION

An object of the present invention is to provide nanoparticles decorated with a positively charged ligand and a negatively charged ligand and having cancer cell-specific cytotoxicity, and the use thereof.

Another object of the present invention is to provide the use of the nanoparticles for cancer cell-specific apoptosis.

Still another object of the present invention is to provide a composition for inducing cancer cell death containing the nanoparticles, and the use thereof.

Yet another object of the present invention is to provide a composition for preventing or treating cancer containing the nanoparticles, and the use thereof.

To achieve the above objects, the present invention provides nanoparticles comprising a nanocore to which a mixture of positively charged ligands and negatively charged ligands has been attached, the nanoparticles exhibiting a positive net surface charge.

The present invention also provides a composition for inducing cancer cell death containing the nanoparticles.

The present invention also provides the use of the nanoparticles for inducing cancer cell death.

The present invention also provides the use of the nanoparticles for preparing a composition for inducing cancer cell death.

The present invention also provides a method for inducing cancer cell death comprising a step of administering the nanoparticles to a subject.

The present invention also provides a pharmaceutical composition for preventing or treating cancer containing the nanoparticles.

The present invention also provides the use of the nanoparticles for preventing or treating cancer.

The present invention also provides the use of the nanoparticles for preparing a pharmaceutical composition for preventing or treating cancer.

The present invention also provides a method for preventing or treating cancer comprising a step of administering the nanoparticles to a subject.

### BRIEF DESCRIPTION OF DRAWINGS

FIG. 1 schematically shows the mechanism by which mixed-charge nanoparticles cancer in lysosomes selectively kill cancer cells. The left and right TEM images confirm the localization of MCNPs in cancer cells and normal cells, and the scale bar is 200 nm. The histogram in the bottom row indicates that the MCNPs of the present invention show cytotoxicity when cells were treated with mixed-charge nanoparticles (200 nM) with a surface ligand ratio of χTMA:χMUA=80:20, and exhibit high cell killing ability against 13 cancer cell lines (right), but exhibit no significant cytotoxicity in 4 normal cell lines (left). Data are presented as mean±SD of three independent experiments.

FIG. 2 shows that the aggregation of mixed-charge nanoparticles depends on protein concentration and pH, but not on ionic strength. FIG. 2 is plotted for a 50 nM nanoparticle solution from the mean distribution for three separate samples measured in triplicate.

FIG. 2a shows the hydrodynamic diameters of nanoparticles with indicated surface ligand ratios in an artificial lysosomal fluid (ALF; Dissolution Technol. 18, 15-28 (2011)) and ALFs supplemented with 1%, 10%, 50% or 75% FBS, at initial pHs of 4.5 (colored bars) and pH 5.5 (black bars).

FIGS. 2b and 2c are maps showing the hydrodynamic diameter of nanoparticles incubated in 10% FBS in water (b) or in 10% FBS in aqueous CaCl₂ solution (c) at the indicated concentrations. In each map, the horizontal axis corresponds to pH and the vertical axis corresponds to the size of the nanoparticles.

FIG. 3 shows the structure and pH-dependent aggregation properties of mixed-charge nanoparticles.

FIG. 3a schematically shows the mixed charge nanoparticles produced in an Example of the present invention. Blue indicates TMA ligands and red indicates MUA ligands.

FIG. 3b is a TEM image of the mixed-charge nanoparticles produced in an Example of the present invention. Scale bar: 20 nm.

FIG. 3c shows the zeta potential of the mixed-charge nanoparticles at pH 7.4. χTMA:χMUA denotes the composition of ligands on the nanoparticle surface. Data are mean±SD values of three independent experiments.

FIG. 3d shows the results of quantifying pH-dependent aggregation of nanoparticles (50 nM) with χTMA:χMUA=100:0 or 80:20 in water supplemented with 10% fetal bovine serum (FBS). The horizontal axis corresponds to pH and the vertical logarithmic axis corresponds to the hydrodynamic diameter measured by dynamic light scattering. Arrows indicate aggregates (DH=about 50 to 100 nm) having the same diameter as the nanoparticle aggregates appearing on the surface of cancer cells.

FIG. 3e shows the size distribution of nanoparticles with χTMA:χMUA=100:0 (right) or 80:20 (left) under each shown condition. The addition of cathepsin D leads to an increase in the aggregate size of 80:20 nanoparticles, but for purely cationic nanoparticles, aggregates grow only marginally or even dissolve upon addition of cathepsin D.

FIGS. 3d and 3e show the average size distributions of three independent experiments.

FIG. 4 shows the aggregation properties of mixed-charge nanoparticles in a cell culture medium.

FIG. 4 shows the hydrodynamic diameter (DH) and zeta potential (ζ) of mixed-charge nanoparticles suspended in cDMEM (supplemented with 10% FBS, purple) and water (pH=7.4, gray). The graph shows the results of one representative experiment.

The inset table shows the mean±SD values of DH and zeta potential measured 10 times. 10% FBS aq represents the data of nanoparticles suspended in water supplemented with 10% FBS. N.A.=Not applicable.

FIG. 5 shows the aggregation of mixed-charge nanoparticles by the proteolytic treatment of protein corona with lysosomal cathepsin D.

FIGS. 5a to 5e show the hydrodynamic diameter (DH) distributions of nanoparticles with ligand ratios of 80:20 (magenta) and 100:0 in mixed solutions of pure ALF (pH4.5) and the substances shown in the respective plots (50% FBS or lysozyme, cathepsin D), determined by DLS. All values consisted of mean distributions of three independent experiments.

FIG. 6 shows the cancer-specific cytotoxic effect mixed-charge nanoparticles depending on the balance of the surface charge.

FIGS. 6a and 6b shows cytotoxicity to human HT 1080 fibrosarcoma versus normal mouse embryonic fibroblasts (MEF) (a), and cytotoxicity against human breast adenocarcinoma MDA-MB-231 versus non-neoplastic epithelial cells MCF-10A (b), when treated with nanoparticles with various ligand ratios at the indicated concentrations (50, 100, and 200 nM).

FIGS. 6c and 6d show the results of comparing cytotoxicity after treatment of each cell type with nanoparticles having ligands at the indicated ratio, in order to determine whether the cancer cell selective cytotoxicity of the mixed-charge nanoparticles is due to the dilution of the surface charge. Gray represents nanoparticles treated with a neutral ligand (C9) to dilute the surface density of cationic charges, and it appears that non-selective cytotoxicity was rather strong. Only 80:20 mixed-charge nanoparticles showed cancer-specific cytotoxicity. The inset plots show the results of cytotoxicity over time when each cell type was treated with 50 nM nanoparticles. All data are presented as mean±SD values of three independent experiments.

FIG. 7 shows the result of confirming the effect of mixed-charge nanoparticles when mouse embryonic fibroblasts (MEFs) were treated with the nanoparticles for a long period of time. FIG. 7 shows cytotoxicity graphs plotted as a function of time for non-tumor mouse embryonic fibroblasts (MEFs) treated with purely cationic nanoparticles (100% TMA, red), purely anionic nanoparticles (100% MUA, green) and mixed-charge nanoparticles with the indicated surface ligand ratios, at indicated concentrations. The mixed-charge nanoparticles showed little cytotoxicity even at high concentrations. Data are presented as mean±SD values of three independent experiments.

FIG. 8 shows the non-selective cytotoxicity of mixed-charge nanoparticles of less than 6 nm. FIG. 8 shows cytotoxicity results confirmed by treating HT 1080 fibrosarcoma cells (24 hours) and non-tumor MEFs (48 hours) with mixed-charge nanoparticles having a smaller diameter. Cytotoxicity was expressed as a function of the concentration of nanoparticles.

The inset plot shows time-dependent cytotoxicity to HT 1080 cells upon treatment with 50 nM small-diameter nanoparticles. In order to distinguish the small-diameter nanoparticles from the mixed-charge nanoparticles of the present invention, an “s” is added to indicate that they are mixed-charge nanoparticles having a small diameter.

The Au core diameter (d), hydrodynamic diameter (DH) and zeta potential of small-diameter nanoparticles are shown in the table. The data shown in the graphs are presented as the mean±SD values of three independent experiments. The values indicated in the table are the mean±SD values of at least 70 nanoparticles.

FIG. 9 shows the results of apoptosis analysis performed by Annexin V staining of cancer cells treated with mixed-charge nanoparticles.

FIGS. 9a and 9b show the results obtained by treating HT 1080 cells (a) and MDA-MB231 cells (b) 50 nM of mixed-charge nanoparticles having the indicated ligand ratios for 48 hours, staining the cells with Alexa Fluor 488 Annexin V conjugate and PI (propidium iodide), and performing flow cytometry. In the flow cytometry plot, the lower right quadrant represents apoptotic cells (AnnexinV+PI−), the lower left quadrant represents viable cells (AnnexinV− PI−), the upper two quadrants represent the number of dead cells (PI+), and the number in each quadrant indicates the percentage of cells in each quadrant/group. The plot inserted in the upper right shows the flow cytometry gating strategy used in the experiment. Data shown are the values of three independent experiments.

FIG. 9c shows the results of performing time-dependent image-based analysis using stained HT-1080 cells. * indicates AnnexinV+ PI− apoptotic cells.

FIG. 9d shows the results of performing image-based analysis of apoptosis in MDA-MB-231 using AnnexinV+PI staining. Images were complicated by extracellular vesicles that were stained positive for phosphatidylserine (PS) around intact cells (AnnexinV+ vesicles marked in green around cells).

MDA-MB-231 and other cancer cells are known to secrete extracellular vesicles containing PS in processes independent of apoptosis. To avoid confusion, only a case in which cells were AnnexinV+/PI− and AnnexinV was bound to the periphery of whole cells was defined as “apoptosis”. The results in this figure indicate that mixed-charge nanoparticles increased the dead cells of MDA-MB-231, whereas apoptotic cells were not observed. It has been shown that cancer cell death was insensitive to cyclosporin A (CA), which inhibits Ca2+-dependent mitochondrial transition pore opening and subsequent cytochrome c release. MDA-MB-231 was also insensitive to apoptosis, and HT1080 was partially sensitive to the Caspase 3/7 inhibitor Ac-DEVD-CHO. Scale bar is 10 μm.

FIG. 10 shows the cellular uptake and intracellular local aggregation of mixed-charge nanoparticles in cancer cells versus normal cells.

FIG. 10a shows the results of determining the amount of internalized gold by ICP-AES after treating cells with mixed-charge nanoparticles or purely cationic nanoparticles (50 nM) for 8 hours (HT1080 vs. MEF) or 16 hours (MD-MB-231 vs. MCF-10A).

FIGS. 10b to 10e show various data on intracellular aggregation of mixed-charge nanoparticles with χTMA:χMUA=80:20 (50 nM) analyzed by dark field microscopy (DFM).

FIG. 10b shows Time-dependent aggregation for mixed-charge nanoparticles. The aggregation was determined by quantifying the average RGB intensity over about 1,000 spots for each time point/cell type after subtracting the scattering signals from the corresponding control cells.

FIGS. 10c and 10d show the results of quantifying the numbers of NP clusters/aggregates (here, called objects) of various sizes based on distinct plasmonic scattering signals over time. Objects were classified as small clusters (with diameters d=about 10 to 100 nm; green in 10e), large clusters (d=100-500 nm; red in 10e), aggregates (d=0.5 to 2 μm; orange in 10e) or very large aggregates (d>2 μm; bright orange in 10e).

FIG. 10e shows the results of confirming the aggregation of mixed-charge nanoparticles in cells with a dark field microscope. Scale bars: 10 μm (main) and 2 μm (inset).

FIG. 10f shows the results of confirming the aggregation of mixed-charge nanoparticles in HT1080 cancer cells by TEM. This is followed by internalization via endocytosis, accumulation in MVBs and massive assembly of crystalline aggregates inside lysosomes concurrent with lysosome swelling. The insets in the rightmost panel show 150 nm² regions with regular NP packing in slower-swelling MDA-MB-231 (n=4, lower right corner) and faster-swelling HT1080 (n=24, upper right corner). Scale bars: 200 nm.

FIG. 10g shows the results of quantifying the number of NPs per lysosome from TEM images (HT1080, n=7; MEF, n=13 lysosomes). Data are presented as box-and-whisker plots; boxes delineate lower and upper quartiles of the data, middle blue lines show median values, grey dots show individual data points, whiskers show minimal and maximal values for each dataset. *P=0.01025, ANOVA, Tukey's post-hoc test.

FIG. 5 shows the impact of intracellular aggregation of mixed-charge nanoparticles on lysosome organelles.

FIGS. 11a and 11b show images of HT1080 (a) and MEF (b) cells treated with the indicated nanoparticles (50 nM) for 6 hours. Lysosomes (red) were labelled with LysoTracker Red and Au NP aggregates (green) were visualized in confocal reflection mode. Similar results were observed in three independent experiments. Scale bars: 10 μm (main images) and 2 μm (insets). The brightness and contrast for individual color channels were adjusted with Adobe Photoshop CS6. Background outside the cells and the few adjacent cells were rendered black for clarity.

FIG. 11c shows the results of quantifying lysosomal nanoparticle aggregation as Pearson's correlation coefficient (PCC)37 between LysoTracker and reflection/NP channels. Data are presented as mean±SD.

FIG. 11d shows lysosomal “swelling” assessed by quantifying the diameters of individual lysosomes in cells treated with 80:20 nanoparticles (50 nM) for 6 hours (HT1080 and MEF) or 24 hours (other cell lines). The data were expressed as a percentage increase in the mean lysosomal diameter of the experimental group compared to the control cells not treated with NPs. The exact numbers of data points are as follows.

(FIG. 11c) HT1080: n=15 cells, MDA-MB-231: n=33 cells, MCF-7: n=11 cells, SKBR3: n=11 cells, MEF: n=13 cells, MCF-10A: n=11 cells, Rat2: n=10 cells, CCD1058SK: n=11 cells.

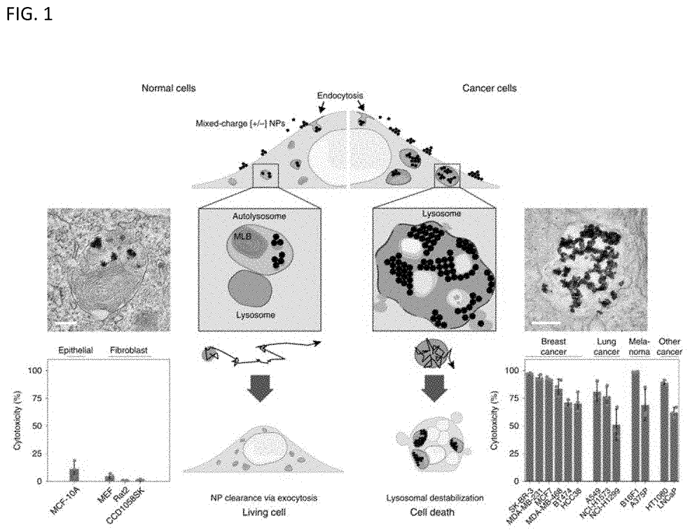

(FIG. 1id) HT1080: n=11 cells, MDA-MB-231: n=5 cells, MCF-10A: n=10 cells, SKBR3: n=11 cells, MEF: n=6 cells, MCF-10A: n=5 cells, Rat2: n=10 cells, CCD1058SK: n=10 cells.

FIGS. 11e and 11f show the results of assessing acridine orange lysosomal membrane integrity after treating HT1080 and MEF (e) with nanoparticles (50 nM) for 6 hours or treating MDA-MB-231 and MCF-10A (f) with nanoparticles for 12 hours. Plots show AO leakage into the cytoplasm quantified as a rise in green fluorescence. Data are presented as mean values. Statistical comparison of values for each NP-treated group and control cells was performed at 120 min. The exact numbers of data points are as follows:

(FIG. 11e) HT1080: control, n=5 independent biological replicates (in FIGS. 11e and 11f, each biological replicate is a time-lapse sequence containing about 20 cells): TMA: n=8, 80:20: n=10, 91:9: n=10, 61:39 n=10. MEF: Control: n=5, TMA: n=5, 80:20: n=5, 91:9: n=5, 61:39: n=5.

(FIG. 11f) MDA-MB-231: Control: n=9, TMA: n=9, 80:20: n=10, 91:9: n=9, 61:39: n=10. MCF-10A: Control: n=5, TMA: n=5, 80:20: n=6, 91:9: n=4, 61:39: n=6.

FIGS. 11g and 11h show cytotoxicity for cancer cells co-treated with 80:20 or 100:0 NPs (100 nM) and lysosomal inhibitors, bafilomycin A (Baf, 100 nM) or chloroquine (Cq, 50 μM), cyclosporine A (CA, 10 μM), an autophagy inhibitor 3-methyladenine (3MA, 5 mM) or an inhibitor of caspases 3/7 Ac-DEVD-CHO (DEVD, 40 μM) for 24 h for HT1080 (g) or 48 h for MDA-MB-231 (h) cells. Control indicates the absence of NP treatment; ‘-’ marks the absence of inhibitors. Data are presented as mean±SD of independent experiments (*P<0.05, **P<0.00001, one-way ANOVA, Tukey's post-hoc test).

FIG. 11i shows mAG-galectin3 puncta/cells before and after exposure to 638 nm laser light after treatment OF MCF7-mAG-gal3 cells for 12 hours as indicated below:

Control=untreated; 100:0=pure TMA NPs; 80:20=TMA:MUA=80:20 mixed-charge nanoparticles; siram=treatment with siramesine (10 μM).

Data are displayed as box-and-whisker plots; boxes delineate the lower and upper quartile of the data, middle blue lines show median values, grey dots show individual data points, and whiskers show minimal and maximal values for each dataset. Two-tailed Student's t-test with unequal variances was used. **p<0.00001 is considered statistically significant. The exact number of each data point is as follows:

(11i) MCF7-mAG-gal3: Control-before: n=4 cells, Control-after: n=14 cells; 100:0-before: n=12 cells, 100:0-after: n=12 cells; 80:20-before: n=17 cells, 80:20-after: n=20 cells; Siramesine-before: n=23 cells, Siramesine-after: n=23 cells.

FIG. 11j shows trajectories of lysosome movements (shown in white) lysosomes (Lysotracker Red, red) and MCF-10A (n=3 videos/cells). Trajectories are superimposed over merged confocal snapshots from movies of lysosomes (Lysotracker Red, red) and [+/−] 80:20 mixed-charge nanoparticles (reflection channel, green). Scale bars=10 μm. Similar results were observed in three cancer cell lines (HT1080, MDA-MB-231, and MCF7) and two normal cell lines (MEF, and MCF-10A). The exact numbers of analyzed images cells are as follows: HT1080: control, n=7; 80:20, n=3; MDA-MB-231: control, n=4; 80:20 n=7; MCF7, control n=3; 80:20, n=3; MEF: control, n=3; 80:20, n=4; MCF-10A: control, n=4; 80:20, n=3.

FIG. 12 shows that mixed-charge nanoparticles accumulate in autophagic vesicles and are excluded from non-tumorigenic cells through exocytosis.

FIG. 12a schematically shows aggregation pathways for cancer (left) versus normal (right) cells. Left: in cancerous cells, mixed-charge nanoparticles first form small clusters inside late endosomes/MVBs; NP clusters then coalesce into larger aggregates inside lysosomes. Autophagosomes (APs) fuse with lysosome, resulting in NP-containing autolysosomes (ALs). NP-induced osmotic flows and imbalance between the fusion/budding events lead to AP-AL ‘swelling’ and their perinuclear clumping, limiting clearance/exocytosis of mixed-charge nanoparticle aggregates from the cancerous cells. Right: in normal cells, mixed-charge nanoparticles accumulate predominantly inside ALs containing MLBs and are rapidly excluded/cleared from cells through exocytosis, as shown in FIG. 12h.

FIG. 12b shows representative TEM images of HT1080 (left) or MEF (right) cells treated with 80:20 nanoparticles for 24 hours. In HT1080 (n=8 cells from two independent experiments), most nanoparticle-containing organelles were non-lamellar and featured intraluminal vesicles (ILVs). In contrast, in MEFs (n=6 cells from two independent experiments), NPs localized to ALs containing MLBs. NP-containing ALs were in contact with early lysosome, resembling the so-called “kiss-and-run” transient vesicle fusion1 (red arrows). Scale bars for TEM images are 1 μm (left, large image) and 200 nm (right, small insets).

FIG. 12c shows representative merged images of APs and ALs marked with eGFP-TagRFP-LC3B autophagy sensor, followed by cell incubation with 80:20 mixed-charge nanoparticles (50 nM) for indicated times (NPs in ALs in magenta; NPs in APs in white).

FIGS. 12d and 12f show the numbers of APs and ALs per cell quantified from images shown in FIG. 12c. Data are presented as mean±SD.

FIG. 12e shows the localization of NPs in HT1080 and MEF cells.

The exact numbers of data points in FIGS. 12c to 12f are as follows:

(12d) HT1080: t=0, n=54 cells; t=12 h, n=24 cells; t=24 h, n=23 cells.

(12e) HT1080: t=2 h, n=13 cells; t=6 h, n=41 cells; t=12 h, n=24 cells; t=24 h, n=23 cells. MEF: t=2 h, n=10 cells; t=6 h, n=16 cells; t=12 h, n=16 cells; t=24 h, n=12 cells.

(12f) MEF: t=0, n=20 cells; t=12 h, n=16 cells; t=24 h, n=12 cells.

The PCC values of the data and statistical analysis in FIG. 12E are as follows.

HT1080: 24 h, PCC (NP/TagRFP ˜APs+ALs)=0.49±0.13; n=23 cells vs. 2 h, 0.18±0.06; n=13 cells, **p<0.00001. 24 h, PCC (NP/eGFP ˜APs)=0.20±0.17; n=23 cells vs. 2 h, 0.07±0.07; n=13 cells,*p=0.003186. Two-tailed Student's t-test with equal variances was used.

MEF: 24 h, PCC (NP/TagRFP)=0.22±0.10; n=12 cells vs. 2 h, PCC=0.04±0.06; n=10 cells,*p=0.000025. 24 h, PCC (NP/eGFP)=−0.02±0.03; n=12 cells vs. 2 h, PCC=0±0.04; n=10 cells, p=0.28228. Two-tailed Student's t-test with equal variances was used.

FIG. 12g shows quantification of lysosomes showing signs of LMP in high-resolution TEM images, such as those shown in FIG. 12b.

FIG. 12h shows the results obtained by incubating MEF and HT1080 cells with 80:20 NPs (50 nM) for 14 hours, and then replacing medium, and quantifying Au exocytosed into the cell culture medium over the indicated time intervals by ICP-AES.

FIG. 12i shows quantification of the Au amounts remaining inside MEF cells following NP internalization (50 nM, 24 hours). In FIGS. 12h and 12i, data are presented as mean±SD of three independent experiments.

FIG. 13 shows the time-dependent cellular uptake of mixed-charge nanoparticles.

Both cancer cells (HT1080 and MDA-MB-231) and normal cells (MEF and MCF-10A) were treated with the indicated types of nanoparticles (50 nM), and the amount of gold internalized in the cells was determined by ICP-AES. Control was not treated with NPs. The vertical axis of each graph represents the kinetics of NP uptake per mg protein in a given sample. Data are presented as mean±SD of three independent experiments.

FIG. 14 shows the results of observing the intracellular aggregation of mixed-charge nanoparticles using a dark field microscope.

FIG. 14a shows the classification of clusters and aggregates of nanoparticles based on the RGB color profiles of the dark field microscopy images. Objects were classified as small clusters (with diameter d=about 10 to 100 nm; green in 10e), large clusters (d=100 to 500 nm; red in 10e), aggregates (d=0.5 to 2 μm; orange in 10e) or very large aggregates (d>2 μm; bright orange in 10e).

FIGS. 14b and 14c are representative DFM images showing time-dependent aggregation of 80:20 mixed charge nanoparticles (50 nM) in MDA-MB-231 adenocarcinoma (b) and non-neoplastic MCF-10A cells (c). Scale bars are 10 μm, and the insets are enlarged views of 5×5 μm² area. Similar results were observed in three independent experiments.

FIG. 15 is a high-resolution TEM image showing packing of crystalline mixed-charge nanoparticles in lysosomes of cancer cells. HT 1080 was treated with 80:20 mixed-charge nanoparticles for 24 hours, and similar results were observed in 23 lysosomes in two independent experiments.

FIG. 16 shows the lysosomal aggregation of mixed-charge nanoparticles in MDA-MB-231 versus MCF-10A cells. FIGS. 16a to 16d are representative confocal images of MDA-MB-3231 (dark, a and c) versus MCF-10A (normal, b and d) treated with various mixed-charge nanoparticles (50 nM) for 12 hours.

Lysosomes (red) were labeled with LysoTracker Red DND-99, and mixed-charge nanoparticle aggregates (green) were visualized in confocal reflection mode. Scale bars are 10 μm (main) and 2 μm (small inset).

FIGS. 16c and 16d show the localization and swelling of 80:20 nanoparticles in lysosomes within 24 hours for MDA-MB-231 and MCF-10A, respectively. For the merged images, the brightness/contrast for individual color channels was adjusted with Adobe Photoshop CS6. The background outside the cells and the few adjacent cells were rendered black.

FIG. 17 shows minimally processed monochromatic individual single channel images of HT1080 and MEF that generated the merged images of FIG. 11. Lysosome organelles labeled with Lysotracker Red DND-99 (red in first row images and merged images) reflection channels show NP aggregates (green in second row images and merged images). In the merged images, the overlap of the two channels is shown in yellow. TD represents a bright field image of the cells, and DIC represents differential interference contrast images of the cells. Scale bar=10 μm. Similar results were observed in three independent experiments.

FIG. 18 shows minimally processed monochromatic individual single channel images of MDA-MB-231 and MCF-10A that generated the merged images of FIG. 16. Lysosome organelles labeled with Lysotracker Red DND-99 (red in first row images and merged images) reflection channels show NP aggregates (green in second row images and merged images). In the merged images, the overlap of the two channels is shown in yellow. TD represents a bright field image of the cells, and DIC represents differential interference contrast images of the cells. Scale bar=10 μm. Similar results were observed in three independent experiments.

FIG. 19 shows the results of analyzing lysosomal swelling through quantification of lysosomal diameters in normal and cancer cells. Lysosomal diameters (μm, vertical axis) in normal cells (MEF, MCF-10A, Rat2, and CCD1058SK) and cancer cells (HT1080, MDA-MB-231, MCF7, and SK-BR-3) were quantified. HT1080 and MEF were treated with 80:20 mixed-charge nanoparticles (50 nM) for 6 hours and the other cell lines were treated for 24 hours (indicated by +) Control was incubated without treatment with nanoparticles (indicated by −). Lysosomal diameters were measured in Lysotracker Red fluorescence images. The mean diameter of cancer lysosomes increased by about 50% when treated with 80:20 mixed-charge nanoparticles (it increased from 0.67 μm to 0.99 μm for HT1080, and increased from 0.67 μm to 1.01 μm for MDA, and a greater increase was observed in MCF7 and SKBR3 breast carcinoma cells). On the other hand, in normal cells, only a low level of increase in diameter was observed even when treated with mixed-charge nanoparticles. Lysosomal diameter data are presented as box and whisker plots. The middle line is the median value, the small rectangles within the boxes represent the mean value, and the whiskers represent the minimum and maximum values. For comparison with the control, a two-tailed Student's test with unequal variances was used. *p<0.05, **p<0.00001 are considered statistically significant. The number of lysosomes (l) is as follows.

MEF: −/Control: l=525 lysosomes, n=7 cells vs. +/80:20 NP: l=759, n=6; *p=0.00002.

MCF-10A: −/Control: l=316, n=10 vs. +/80:20 NP: l=340, n=12; p=0.05198.

Rat2: −/Control: l=689, n=12 vs. +/80:20 NP: 1=787, n=10; p=0.60262.

CCD1058SK: −/Control: l=2337, n=10 vs. +/80:20: l=1025, n=10; **p<0.00001.

HT1080: −/Control: l=707, n=6 vs. +/80:20 NP: 1=539, n=11; **p<0.00001.

MDA-MB-231: −/Control: l=418, n=16 vs. +/80:20 NP: l=235, n=10; **p<0.00001.

MCF7: −/Control: l=250, n=11 vs. +/80:20 NP: 1=239, n=11, **p<0.00001.

SK-BR-3: −/Control: l=233, n=10 vs. +/80:20 NP: l=195, n=10; **p<0.00001.

FIG. 20 shows the aggregation of lysosomal nanoparticles and lysosomal swelling with time. FIGS. 20a and 20b show the results of quantifying the co-localization of NPs and acidic endo-lysosomal organelles in HT1080 and MEF (a), and MDA-MB-231 and MCF-10A (b) using Pearson's correlation coefficient (PCC) between LysoTracker and reflection/NP channels (see FIGS. 11a and 16). Data are presented as mean±SD values.

The exact numbers of data points are as follows:

HT1080:

TMA: t=1 h, n=11 cells; t=2 h, n=17 cells; t=4 h, n=14 cells, t=6 h, n=11 cells;

91:9: t=1 h, n=11 cells; t=2 h, n=12 cells; t=4 h, n=10 cells, t=6 h, n=9 cells;

80:20 t=1 h, n=10 cells; t=2 h, n=10 cells; t=4 h, n=10 cells, t=6 h, n=15 cells;

61:39 t=1 h, n=15 cells; t=2 h, n=18 cells; t=4 h, n=17 cells, t=6 h, n=12 cells.

MEF:

TMA: t=1 h, n=9 cells; t=2 h, n=12 cells; t=4 h, n=12 cells, t=6 h, n=12 cells;

91:9: t=1 h, n=9 cells; t=2 h, n=7 cells; t=4 h, n=12 cells, t=6 h, n=12 cells;

80:20: t=1 h, n=7 cells; t=2 h, n=16 cells; t=4 h, n=16 cells, t=6 h, n=13 cells;

61:39: t=1 h, n=11 cells; t=2 h, n=11 cells; t=4 h, n=12 cells, t=6 h, n=6 cells.

MDA-MB-231:

TMA: t=1 h, n=8 cells; t=4 h, n=20 cells; t=12 h, n=12 cells, t=24 h, n=27 cells;

91:9: t=1 h, n=15 cells; t=4 h, n=19 cells; t=12 h, n=15 cells, t=24 h, n=24 cells;

80:20: t=1 h, n=12 cells; t=4 h, n=16 cells; t=12 h, n=12 cells, t=24 h, n=33 cells;

61:39: t=1 h, n=6 cells; t=4 h, n=10 cells; t=12 h, n=15 cells, t=24 h, n=13 cells.

MCF-10A:

TMA: t=1 h, n=6 cells; t=4 h, n=11 cells; t=12 h, n=8 cells, t=24 h, n=21 cells;

91:9: t=1 h, n=9 cells; t=4 h, n=9 cells; t=12 h, n=7 cells, t=24 h, n=15 cells;

80:20: t=1 h, n=4 cells; t=4 h, n=4 cells; t=12 h, n=6 cells, t=24 h, n=11 cells;

61:39: t=1 h, n=3 cells; t=4 h, n=8 cells; t=12 h, n=8 cells, t=24 h, n=12 cells.

FIGS. 20c and 20d show lysosomal “swelling” data assessed by quantifying the diameters of individual lysosomes in cells treated with mixed-charge nanoparticles (50 nM). The data are expressed as a percentage increase in the mean lysosomal diameter of the experimental group compared to the control cells not treated with NPs. n=5 for each cell and treatment type. Data are presented as mean±SD values.

FIG. 21 shows the results of statistical analysis of data obtained through an acridine orange lysosomal membrane integrity test. Green fluorescence intensity values at a laser exposure time of 120 seconds were compared using one-way ANOVA and Tukey's post-hoc test. The p values for all comparisons are described in the table. *p<0.05, **p<0.00001 are considered significantly different.

The exact numbers of data points are as follows:

HT1080: Control (Cont): n=5 independent biological replicate (each biological replicate is a time-lapse sequence/movie, and each movie records about 20 cells); TMA: n=8; 80:20: n=10; 91:9: n=10; 61:39: n=10.

MEF: Control: n=5; TMA: n=5; 80:20: n=5; 91:9 n=5; 61:39: n=5.

MDA-MB-231: Control: n=9; TMA: n=9; 80:20: n=10; 91:9: n=9; 61:39: n=10.

MCF-10A: Control: n=5; TMA: n=5; 80:20: n=6; 91:9: n=4; 61:39: n=6.

FIG. 22 shows the results of testing the lysosomal membrane permeability using galectin puncta assay.

In FIG. 22a, control was untreated, and MCF7-mAG-gal3 cells (MCF7=adenocarcinoma) or U2OS-mCherry-gal3 cells (U2OS=osteosarcoma) were treated with 100:0 or 80:20 NPs (100 nM) or siramesine (10 μM) for 12 hours. The number of galectin3 puncta/cell was counted for about 30 seconds before (full) and after (empty) exposure to 638 nm laser. Data are displayed as box-and-whisker plots; boxes delineate the lower and upper quartiles of the data, middle lines show median values, circles show individual data points, and whiskers show minimal and maximal values for each dataset.

FIG. 22b depicts images showing the results of quantifying MCF7-mAG-gal3 cells before and after exposure to 638 nm laser light. Scale bar=10 μm. Similar results were observed in two independent experiments. The exact numbers of data points are as follows:

MCF7-mAG-gal3: Control-before: n=14 cells, Control-after: n=14 cells; 100:0-before n=12 cells, 100:0-after n=12 cells; 80:20-before n=17 cells, 80:20-after n=20 cells; Siramesine-before n=23 cells, Siramesine-after n=23 cells.

U2OS-mCherry-gal3: Control-before: n=10 cells, Control-after: n=10 cells; 100:0-before: n=9 cells, 100:0-after: n=9 cells; 80:20-before: n=10 cells, 80:20-after: n=10 cells; Siramesine-before: n=20 cells.

FIG. 23 shows the effect of co-treatment with nanoparticles and lysosomal and autophagy inhibitors in non-tumor cells. Cells were co-treated with 80:20 mixed-charge nanoparticles (magenta) or purely cationic (TMA 100%) nanoparticles (orange) (100 nM) and bafilomycin A (Baf, 100 nM), chloroquine (Cq, 50 μM), or an autophagy inhibitor 3-methyladenine (3MA, 5 mM). Control indicates cells treated only with inhibitors without NP treatment; ‘−’ marks the absence of inhibitors. Data are presented as mean±SD of independent experiments. Data are presented as mean±SD of three independent experiments.

FIG. 24 shows representative confocal images (snapshots from movies) of HT1080 versus MEF cells showing the effect of mixed-charge nanoparticles on lysosomal movement trajectories. Control was untreated and each cell type was treated with purely cationic NPs (TMA 100%) or 80:20 mixed-charge nanoparticles for 8 hours. Lysosomes (red) were labeled with LysoTracker Red DND-99 and NP aggregates (green) were visualized in confocal reflection mode. Lysosomal movement was tracked and analyzed using NIS-Elements (Nikon) software, and the lysosomal trajectories (white) were displayed on the snapshot image from each movie. For the control (top panel), lysosomal trajectories were not different between cancer cells and normal cells, and all the cell types featured random/“jiggly” juxtanuclear and directional peripheral trajectories. When treated with mixed-charge nanoparticles, swelling occurred inside the cancer cell lysosomes, and movement was significantly delayed. Peripheral lysosomes disappeared and the remaining trajectories were random/“jiggly” (lower left panel). In contrast, when treated with mixed-charge nanoparticles, the movement of normal lysosomes in normal cells was fast, directional, and not inhibited (lower right panel). When treated with pure TMA nanoparticles, the movement of cancer cell lysosomes was dynamically maintained with peripheral directional trajectories (middle panel). Scale bar=10 μm. The exact numbers of data points in the image/cell analysis are as follows:

HT1080: Control, n=7; 80:20, n=3; 100:0, n=3;

MEF: Control, n=3; 80:20, n=4; 100:0, n=3.

FIG. 25 shows the results of analyzing the spatial distribution of lysosomes between the nuclear boundary (P=0) and the cell periphery (P=1) in units of individual cells with reference to FIG. 11.

FIG. 25a shows a gray scale inverted LysoTracker RED fluorescence channel of single cells superimposed on the indicated nuclear boundary (blue) and cell periphery (orange) (the darker it is, the stronger the fluorescence). The locations of the nuclear and cell boundaries were manually marked on the DIC channel and applied to the red channel image. For a given point (T), the light OT (green) is cast from the nuclear center of mass O (yellow), and OT intersects the nuclear boundary at point A and the cell periphery at point B. The actually measured perinuclearity (P) for the point T is defined such that the interval OA is mapped to the [−1,0] range of P and the interval AB is mapped to the [0,1] interval of P.

In FIG. 25b, isolines of different levels of P were calculated from the cell image of FIG. 25a and displayed as outlines. P=1 at the cell periphery, P=0 at the nuclear boundary, and P=−1 at the nucleus center.

FIG. 26c shows the results obtained by treating cells with 80:20 mixed-charge nanoparticles (50 nM/6 hours for HT-1080 and MEF, and 24 hours for all other cell lines), labeling lysosomes with Lysotracker Red, acquiring a confocal microscope image, and analyzing the spatial distribution. The distribution I(P) obtained for individual cells was combined into a single distribution I(P) for each cell line and displayed as a violin plot. The middle line is the median value, and the dotted line represents the 3rd and 1st quartiles of the data. For statistical analysis of perinuclearity, the mean perinuclearity for each cell was calculated, and values between two samples were compared using Kolmogorov-Smirnov (K-S) test, Mann-Whitney (M-W) U-test, and two-tailed Student's t-test with equal variances. There was no significant difference in all cell lines. The exact values for each cell are as follows.

Cancer:

SKBR3 (80:20: n=12 cells; Control: n=11, p-values: K-S 0.551, M-W 0.259, t-test 0.580);

MCF7 (80:20: n=11; Control: n=12, p-values: K-S 0.985, M-W 0.448, t-test 0.779);

MDAMB231 (80:20: n=14; Control: n=11, p-values: K-S 0.560, M-W 0.214, t-test 0.399);

HT1080 (80:20: n=15; Control: n=6, p-values: K-S 0.301, M-W 0.129, t-test 0.215).

NORMAL:

MCF-10A (80:20: n=9; Control: n=7, p-values: K-S 0.735, M-W 0.170, t-test 0.190);

MEF (80:20: n=12; Control: n=9, p-values: K-S 0.051, M-W 0.030, t-test 0.036);

Rat2 (80:20: n=10; Control: n=12, p-values: K-S 0.036, M-W 0.022, t-test 0.071);

CCD1058sk (80:20: n=10; Control: n=10, p-values: K-S 0.313, M-W 0.061, t-test 0.109).

FIG. 26 shows TEM images after treating cancer cells and normal cells with nanoparticles. The exact numbers of cells analyzed were as follows: (a) n=2 cells; (b) n=3 cells; (c) n=5 cells; (d) n=2 cells (two independent experiments).

FIGS. 26a and 26b are TEM images after treating HT 1080 cells with 50 nM purely cationic nanoparticles (TMA 100%) for 6 hours. Nanoparticles are internalized into cells through loose aggregation on the cell surface, direct membrane penetration (green arrowhead), and cytoplasmic aggregation (green arrow). Purely cationic nanoparticles have limited aggregation in lysosomal-like organelles, and yellow arrowheads indicate potential endosomal/lysosomal escape into the cytosol.

FIGS. 26c and 26d show the normal microstructure of HT1080 (c) and MEF (d) when not treated with NPs: AL=autolysosome, MLB=multilamellar body, LV=lysosomal vacuole.

FIG. 27 shows the accumulation of mixed-charge nanoparticles in autophagic vesicles of MDA-MB-231 and MCF-10A cells. Autophagosomes (APs) and autolysosomes (ALs) were labeled with the eGFP-TagRFP-LC3B autophagy sensor, and each cell type was incubated with 80:20 mixed-charge nanoparticles for the indicated time.

FIG. 27a shows a representative merged image. AP is shown in yellow and AL in red; NP/AL overlap in magenta and NP/AP overlap in white. Scale bar=10 μm.

FIGS. 27b and 27d show the results of quantifying the number of vesicles per cell in the image of FIG. 27a, and the plot of FIG. 27c shows the percentage of cells containing NPs localized to yellow (AP), red (AL), two types of vesicles (AP+AL) or vesicles not localized to any of the two types of vesicles. The rate of aggregation into autophagic organelles was different, but the localization pattern was similar. In FIGS. 27b and 27d, data are presented as mean±SD values. The exact numbers of data points are as follows:

(27b) MDA-MB-231: t=0, n=32 cells; t=12 h, n=22 cells; t=24 h, n=22 cells.

(27c) MDA-MB-231: t=2 h, n=20 cells; t=6 h, n=19 cells; t=12 h, n=22 cells; t=24 h, n=22 cells.

MCF-10A: t=12 h, n=26 cells; t=24 h, n=24 cells; t=48 h, n=14 cells.

(27d) MCF-10A: t=0, n=27 cells; t=12 h, n=16 cells; t=24 h, n=24 cells; t=48 h, n=14 cells.

MDA-MB-231: t=24 h, PCC (NP/TagRFP ˜APs+ALs)=0.31±0.12; n=22 cells vs. t=2 h, 0.04±0.08; n=20 cells, **p<0.00001. t=24 h, PCC (NP/eGFP ˜APs)=0.08±0.09; n=22 cells vs. t=2 h, PCC=0±0.05; n=20 cells,*p=0.000859 (two-tailed Student's t-test with equal variances).

MCF10A: t=48 h, PCC (NP/TagRFP)=0.37±0.11; n=14 cells vs. t=12 h, PCC=0.20±0.09; n=26 cells, **p<0.00001. t=48 h, PCC (NP/eGFP)=0.01±0.02; n=14 cells vs. t=12 h, PCC=0.02±0.03; n=26 cells, p=0.443353 (two-tailed Student's t-test with equal variances).

FIG. 28 shows the clearance of internalized mixed-charge nanoparticles in normal cells.

In FIGS. 28a and 28b, MEF cells (24 hours) or MCF-10A cells (48 hours) were incubated with 80:20 mixed-charge nanoparticles or purely cationic nanoparticles (TMA 100%), and the medium was replaced so that the cells could release/clear the internalized NPs into the medium (t=0). The amount of Au remaining inside the cells was quantified using ICP-AES at the indicated times (t=0 corresponds to immediately after 24 hours or 48 hours of incubation in each cell line). Data are presented as mean±SD of three independent experiments.

FIGS. 28c and 28d are representative images of MEF cells (c) or MCF-10A cells (d) at each time point. NP aggregates were observed with confocal reflection (green) and differential interference contrast (DIC). Scale bar=10 μm. Similar results were observed in the following number of experiments/images: (c) MEF: No NPs: n=3 imaged fields of view; t=0 day, n=32; t=2 days, n=12; t=5 days, n=14; t=6 days, n=15 (two independent experiments). (d) MCF-10A: No NPs: n=8; 0 day, n=23; 2 days, n=21; 4 days, n=18; 6 days, n=20 (three independent experiments). The clearance of mixed charge nanoparticles (80:20) identified using confocal reflection microscopy shown in FIGS. 28c and 28d is consistent with the ICP-AES results shown in FIGS. 28a and 28b.

FIG. 29 shows the effect of mixed charge nanoparticles on protein corona.

FIG. 29a shows the hydrodynamic diameters of 80:20 mixed charge nanoparticles (magenta) and purely cationic (100:0, orange) nanoparticles. Similar results were observed in water, water containing 10% FBS, and DMEM. Data are presented as mean±SD of three independent experiments.

FIG. 29b shows that the positive zeta potentials of 80:20 mixed-charge nanoparticles and purely cationic nanoparticles change to negative in the presence of serum protein (water+10% FBS) consistent with protein corona formation. Data are presented as mean±SD of three independent experiments.

FIG. 29c shows a Coomassie blue stained SDS-PAGE gel obtained by treating pure TMA nanoparticles (100:0), mixed-charge nanoparticles (80:20), or pure MUA nanoparticles (0:100) with 50% FBS for 1 hour and quantifying the amount of attached serum protein by BCA (bicinchoninic acid assay). The total protein amount of protein corona is as follows (three independent experiments):

TMA: 479.3±47.6 μg/mL;

80:20: 305.7±88.2 μg/mL;

MUA: 288.7±26.4 μg/mL

FIG. 30 shows the role of protein corona in the cellular uptake of mixed-charge nanoparticles.

FIGS. 30a and 30e show the results obtained by treating HT 1080(a) and MEF cells with the indicated nanoparticles, incubating the cells in complete cell culture medium (cDMEM, FBS) or free-FBS medium (DMEM) for 8 hours, and quantifying the amount of Au nanoparticles internalized into the cells by ICP-AES. Data are presented as mean±SD values of three independent experiments.

FIGS. 30b and 30f show the degree of co-localization of 50 nM 80:20 mixed charge nanoparticles (green) and lysosomes (red) in HT 1080 (b) or MEF (f) cells in cDMEM with or without FBS. Scale bar=10 μm. The numbers analyzed in each cell type are as follows:

HT 1080: cDMEM: n=11, DMEM: n=17;

MEF: cDMEM: n=6, DMEM: n=14.

FIGS. 30c and 30g show the results of quantifying the lysosomal diameter (DL) for the images shown in FIGS. 30b and 30f. Lysosomal diameter data are presented as box-and-whisker plots, the upper limit of the box represents the third quartile, the lower limit of the box represents the first quartile, the middle line represents the median, the small rectangle inside the box represents the mean value, and the whiskers represent minimal and maximal values for each dataset. The blue dotted line represents the mean diameter of control cell lysosomes.

The lysosomal diameter in HT1080 increased similarly in DMEM (about 65% increase) and cDMEM (about 49% increase), whereas a slight increase in lysosomal diameter was observed in MEF (DMEM: about 16%, cDMEM about 8%). The exact numbers of data points are as follows:

(30c) HT1080: cDMEM: l=539 lysosomes, n=11 cells; DMEM: l=367, n=12;

(30g) MEF: cDMEM: l=759, n=6; DMEM: l=1540, n=10.

(30d, 30h) Cells were treated and incubated with 80:20 mixed-charge nanoparticles (magenta) or 100:0 purely cationic nanoparticles (orange) in the presence (cDMEM) or absence (DMEM) of FBS for 24 hours. The black line represents untreated control. The selectivity of cytotoxicity was evaluated by the Selectivity Index (SI), and in the case of 80:20 mixed-charge nanoparticles, the selectivity of cytotoxicity decreased from SI=55.6 in cDMEM to SI=6.8 in DMEM. Data are presented as mean±SD of three independent experiments.

FIG. 31 shows the lysosomal swelling effect of cancer cell-specific mixed-charge nanoparticles and the effects of a protease inhibitor and rapamycin on cytotoxicity.

FIG. 31a shows the results of confirming lysosomes after treating cells with a cysteine and serine protease leupeptin inhibitor (150 μM), a serine protease inhibitor E64 (100 μM), or rapamycin (10 μM), followed by treatment with 80:20 mixed-charge nanoparticles (50 nM) for 24 hours. Lysosomes (red) were labeled with LysoTracker Red DND-99, and NP aggregates (green) were visualized in confocal reflection mode. Scale bar=10 μm. Similar results were observed in two independent experiments.

FIG. 31b shows the lysosomal diameter of HT 1080 cells treated as shown in FIG. 31a. Lysosomal diameter data are presented as box-and-whisker plots; boxes delineate lower and upper quartiles of the data, middle lines show median values, small rectangles within the boxes show mean values, and whiskers show minimal and maximal values for each dataset. The exact numbers of data points/cells analyzed are as follows:

−/80:20: l=106, n=5; Leupeptin/80:20: l=178, n=5; E64/80:20: l=378, n=5; Rapamycin/80:20: l=342, n=5. **p<0.00001. (One-way (ANOVA) and Tukey's post-hoc test).

FIG. 31c shows cytotoxicity (% of dead cells) to HT1080 (magenta bar) versus MEF (black bar). Data are presented as mean±SD of three independent experiments. *p<0.05, **p<0.00001. (One-way (ANOVA) and Tukey's post-hoc test). When cells were treated with leupeptin, lysosomal swelling and cytotoxicity induced by 80:20 mixed-charge nanoparticles were partially reduced, and when cells were treated with E64, the degree of reduction was low. Similarly, treatment with a low concentration of rapamycin reduced lysosomal swelling and cytotoxicity, suggesting that 80:20 mixed-charge nanoparticles could interfere with mucolipin-I function.

FIG. 32 shows the effect of mixed-charge nanoparticles on cellular acid sphingomyelinase and acid ceramidase activities.

FIG. 32a schematically shows acid sphingomyelinase (ASMase) and acid ceramidase (ACDase) lysosomal sphingolipid metabolic pathways.

FIG. 32b shows ASMase activity in lysates of HT 1080 and MEF cells treated with 5 μM siramesine (Siram, gray bars), 100 nM 80:20 mixed-charge nanoparticles (magenta bars), or 100 nM purely cationic nanoparticles (TMA/100:0) (orange bars) for 18 hours. 1×10⁶ cells were manually homogenized by addition of additional protease inhibitor cocktail (Sigma-Aldrich, #P8340) on ice in the buffer provided, and enzyme activity was measured immediately. Data are presented as mean±S.E.M values. The exact numbers in independent experiments are as follows:

HT1080: control, n=3 independent experiments; 80:20, n=4; 100:0, n=3; Siram., n=4;

MEF: control, n=3; 80:20, n=4; 100:0, n=3; Siram., n=4.

* means statistically significant difference compared to control. *p<0.05, (One-way (ANOVA) and Tukey's post-hoc test).

FIG. 32c shows ACDase concentrations in cell lysates (2×10⁶ cells/mL) untreated or treated with 100 nM 80:20 mixed-charge nanoparticles (magenta and black bars, respectively) for 18 hours. A sandwich ELISA kit was used according to the manufacturer's protocol (LifeSpan BioSciences, #LS-F6337). Technical replicates of two independent experiments are n=8, and data are presented as mean±SD.

* means statistically significant difference compared to control. *p<0.05, (One-way (ANOVA) and Tukey's post-hoc test).

FIG. 33 shows the results of analyzing the difference in lysosomal protein when normal cells or cancer cells were treated with mixed-charge nanoparticles. MCF10A (=normal) and MCF7 (=cancer) cells were treated with 100 nM mixed-charge nanoparticles with χTMA:χMUA=80:20 for 24 hours, and intact lysosomes were isolated using a lysosomal enrichment kit (Thermo Scientific, cat #89839).

FIG. 33a shows the results of collecting and analyzing two lysosomal fractions from cells treated with nanoparticles. An upper fraction containing lysosomes with little or no nanoparticles (Lyso, cyan) and a lower fraction containing lysosomes with internalized nanoparticles (NPs, magenta) were observed. For a control group, cells were cultured in the same manner and only an upper (Lyso) fraction was collected and analyzed without NP treatment.

FIG. 33b shows an SDS-PAGE gel obtained by staining the lysosomal protein sample (5 μg protein/well) prepared as shown in FIG. 33a with Coomassie blue. Similar results were observed in three independent experiments. The darker background “smearing” in the MCF10A/NPs fraction is believed to be due to glycosylation and/or other post-translational modifications of lysosomal protein. All fractions, including the NP fraction at the bottom, were found to contain lysosomes by Western blotting for LAMP2 protein.

FIGS. 33c and 33d are plots obtained by plotting equal-length protein migration profiles for the samples shown in FIG. 33b using Gwyddion v2.52 without background removal. The horizontal axis shows the molecular mass of the band as determined from the migration profile of the standard protein ladder (std, first lane of FIG. 33b). The locations of the major protein bands were manually identified (corresponding to the peaks in the profile and detected with an error of approximately ±2 kDa). Control samples were compared to NP-treated samples (for clarity, bands of 80:20/Lyso+80:20/NPs were combined to represent all lysosomes in the samples).

FIG. 33e shows the major changes in protein levels when comparing control samples to NP-treated samples. Arrows indicate changes in peak intensity for NP-treated samples compared to control samples. FIG. 33e shows an at least 20% increase or decrease in each base-peak height. If bands of specific molecular mass were present in both the Lyso and LPs fractions, the intensities were summed and compared cumulatively with the intensities of the control bands. This figure means that mixed-charge nanoparticles induce more distinct changes in lysosomal proteins in cancer cells than in normal cells.

FIG. 34 shows the differential regulation of the lysosomal mTORC1 complex by mixed-charge nanoparticles in normal cells versus cancer cells.

FIG. 34a schematically shows the possible results of inhibition of mTORC1 signaling due to activation of the mTORC1 protein complex in normal cells and displacement of key components in the lysosomal membrane of cancer cells.

FIG. 34b shows the relative amounts of LAMP2, RagC and mTOR (major component of mTORC1) proteins, determined by Western blotting of the lysosomal fractions (Lyso, MPs) for control cells (not treated with mixed-charge nanoparticles treatment, Control) and cells (MCF-10A or MCF7) treated with 100 nM 80:20 mixed-charge nanoparticles. The presence of LAMP2 protein in all samples indicated that lysosomes were contained in all fractions including the NPs fraction at the ‘bottom’. RagC (about 50 kDa) and mTORα (about 280 kDa) proteins were detached from the surface of cancer lysosomes (MCF7:NPs) containing 80:20 mixed-charge nanoparticles. In contrast, the amounts of RagC (about 50 kDa) and mTORα (about 280 kDa) rather increased on the surface of the lysosomes (MCF-10A: Lyso and NPs) of normal cells. The importance of the mTORβ isoform (identified by the same antibody as that for mTORα) was not fully found, but was included for completeness. Band intensities were quantified in 16-bit digital images by densitometry using ImageJ software and normalized to the intensity of the control lane for each cell type (numbered below the bands). Similar results were observed in three independent experiments.

FIG. 35 shows the results of estimating the number of nanoparticles inside a single lysosome.

FIG. 35a shows raw TEM images of lysosomal slices to estimate the volume fraction of nanoparticles in the lysosome. The TEM image was divided into the following three regions: a region free of nanoparticles (light lime color), a region containing about 3 layers packed with nanoparticles (green), and a region containing a thick volume packed with NPs (purple). The darkest region was assumed to have the same thickness as a microtome slice (80 nm). Also, for the sake of simplification, it was assumed that NPs were dense.

FIGS. 35b and 35c show the derivation of a geometric correction factor for the effective radius of the lysosome.

FIGS. 35d to 35f show data for HT 1080, MDA-MB-231 and MEF incubated with 50 nM 80:20 mixed-charge nanoparticles for 24 hours from left to right. The meaning of each element in box plots is as follows: whiskers: minimal and maximal values for each dataset, box edges: 25% and 70% percentiles, blue lines: median values. Green dots show overlapping data points. The left axis of FIG. 35D shows the effective diameter of a lysosome (diameter of a circle having the same area as a given lysosome) in a microtome cross-section taken by TEM. The right axis differs from the left axis by a 4/π coefficient, and corresponds to the actual estimated diameter of the lysosome.

FIG. 35E shows the number of nanoparticles in a microtome section of the lysosome (see FIG. 35a).

FIG. 35f shows the estimated number of nanoparticles inside a single lysosome. Lysosomes of cells (cancer: HT1080, normal: MEF) incubated with 50 nM 80:20 mixed-charge nanoparticles for 24 hours were used for analysis. The sizes of the lysosomal samples in FIGS. 35d to 35f are as follows. n=7 (HT1080), n=10 (MDA-MB-231), n=13 (MEF).

FIG. 36 shows the effect of osmotic pressure on the destruction of cancer lysosomes by mixed-charge nanoparticles.

The osmotic pressure across the lysosomal membrane due to the presence of AuNPs was calculated for various volume fractions (ratio of total volume of AuNPs in the lysosome to the volume of the lysosome, q) and indicated by blue ribbons. The side of the blue ribbon corresponds to the uncertainty of the ambient osmotic pressure (280 to 320 mOsm/L). The gray horizontal dotted line indicates the minimum pressure (1.4 atm, estimated as 4σ_{c}/D_{c} using typical membrane tension σ_{c}=0.4 mN/m for long-term stability and a lysosomal diameter of about 1 μm) that can induce lysosomal destruction in normal cells, as reported in J. Mater. Chem. B 2, 3480-3489 (2014). The range of pressure that induces destruction of cancer lysosomes is lower than the pressure required to destroy lysosomes in normal cells.

The lower panel shows box plots of the nanoparticle volume fractions of individual lysosomes estimated from TEM images for HT 1080 after 6 and 24 hours and MDA-MB-231 cells after 24 hours. In the whisker-and-box plots, the boxes delineate the lower and upper quartiles of the data, the purple line represents the median value, and the whiskers correspond to the minimal and maximal volume fractions for each dataset. Sample sizes were as follows: n=10 lysosomes (HT1080, 6 h), n=7 lysosomes (HT1080, 24 h), and n=10 lysosomes (MDA-MB-231, 24 h). Individual results are indicated by superimposed dots on box plots, and the orange brackets indicate the volume fraction that could be achieved if all nanoparticles added to the cell culture were internalized into lysosomes. The blue diamond in the left corner shows the lower limit of the volume fraction in HT cells after 6 hours, based on the measured uptake of nanoparticles (ICP-AES, see FIG. 13). The right edge of the horizontal axis represents the largest possible volume fraction for a sphere (η=π/(3√2)).

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Any concentration range, percentage range, or integer range described herein may be understood as any integer value within the stated range, and, where appropriate, fractions thereof (e.g., 1/10 and 1/100 of an integer).

Charged polymers or nanoparticles have continued to attract a lot of interest and research due to their high potential for use in medicine and pharmaceuticals due to their electrical properties and direct role in vivo. However, due to their electrical properties in the complex microenvironment of cells, in designing nanoparticles for medical and pharmaceutical purposes, it is very difficult to predict or control target cell selectivity, intracellular localization, etc., and thus their use is very limited. For example, negatively charged nanoparticles are not readily uptaken by adherent mammalian cells except phagocytes, and positively charged nanoparticles are difficult to use for medical and pharmaceutical applications due to their non-selective cytotoxicity.

The present inventors have continuously studied the biological properties of mixed-charge nanoparticles (MCNPs) modified with mixed ligands (mixed at various ratios) bearing opposite charges, and have reported pH-dependent precipitation and in vitro crystallization thereof (Front. Physiol. 4, 370, 2013). At the same time as this report of the present inventors, Liu, X. S. et al. reported that MCNPs can aggregate in cancer cells at an acidic pH, the aggregates can be uptaken into cancer cells, and they can be used for cancer cell-targeted drug delivery or as biosensors (Small 2014, 10, 4230-4242; ACS Nano 2013, 7, 6244-6257). Based on the existing application of gold nanoparticles to tumor treatment, Liu, X. S. et al. applied for a patent for the use of MCNPs in combination with magnetic/near-infrared thermotherapy for tumor treatment (CN 102989016 A). However, in the above document, Liu, X. S. et al. use only the cancer cell localization ability of the MCNPs of the present invention, and do not disclose the use of the MCNPs themselves for cancer cell-specific death at all.

In an example of the present invention, in addition to the simple tumor-targeting ability of MCNPs, MCNPs having tumor-specific cytotoxicity were designed and produced by controlling the ratio between mixed ligands, and cancer cell-specific cytotoxicity thereof was evaluated. As a result, it was confirmed that MCNPs mixed with ligands to have a positive net surface charge were specifically localized and crystallized in the lysosomes of cancer cells, resulting in cancer cell death.

In another example of the present invention, it was confirmed that, although the mixed-charge nanoparticles of the present invention bear a positive surface charge, they show no significant cytotoxicity to normal cells and show cancer cell-specific cytotoxicity, unlike conventional cationic nanoparticles that exhibited non-specific cytotoxicity. In addition, it was confirmed that this cancer cell-specific apoptosis property is not caused by dilution of a positive charge, and that the MCNPs have a novel cytotoxic mechanism that induces lysosomal swelling and lysosomal membrane permeabilization (LMP), resulting in LMP-mediated lysosomal cell death (LCD), unlike the cytotoxic mechanism of conventional cationic nanoparticles (FIG. 1).

In non-tumor cells, the mixed-charge nanoparticles (MCNPs) of the present invention accumulate as small aggregates in late autolysosomes with multilamellar bodies (MLBs), whereas in cancer cells, they form very large aggregates in multivesicular bodies (MVBs). In particular, lysosomes can be characterized in that they completely fuse with MVBs to form endolysosomes, and induce supercrystallization of MCNPs, resulting in lysosomal destabilization and cell death.

Therefore, in one aspect, the present invention is directed to mixed-charge nanoparticles comprising a nanocore to which a mixture of a positively charged ligand and a negatively charged ligand has been attached, the nanoparticles having a positive net surface charge.

As used herein, the term “nanocore” refers to a molecule constituting a central nucleus in the synthesis of nanoparticles, and in general, the size, shape and main properties of nanoparticles are determined by the nanocore. Examples of the nanocore include, but are not limited to, metal nanocores (e.g., gold, silver, etc.), semiconductor quantum dots (e.g., CdSe, CdTe, PbS, perovskite (methylammonium, cesium halide)), and transition metal oxides (iron oxide, cobalt oxide, silicon oxide, etc.), and also include any type of nanocore suitable for use in the present invention.

In the present invention, the nanocore may be selected from the group consisting of metal nanocores, semiconductor quantum dots, and transition metal oxides. Preferably, the nanocore may comprise a metal or metal oxide nanocore, more preferably a gold, iron oxide or silicon oxide nanocore, most preferably a gold nanocore.

As used herein, the term “mixed-charge nanoparticles” refers to nanoparticles having a nanocore to which a mixture of a positively charged ligand and a negatively charged ligand has been attached, and is used interchangeably with the term as “MCNPs (Mixed-Charged Nano-Particles)”. In the present invention, the ratio between the surface ligands attached to the nanocore of the mixed-charge nanoparticles is described in the form of “X:Y,” and unless otherwise specified, X in the ratio denotes the positively-charged ligand (TMA in the examples and drawings), and Y denotes the negatively charged ligand (MUA in the examples and drawings).

In the present invention, the size, surface charge, and ligand ratio of the mixed-charge nanoparticles were considered the most important factors in the cancer cell-specific cytotoxicity of the nanoparticles.

The size of the nanoparticles may be mainly determined by the diameter of the nanocore. In one example of the present invention, it was expected that nanoparticles having a hydrodynamic diameter (DH) of about 6 nm to 20 nm would be suitable. To this end, mixed-charge nanoparticles having an average hydrodynamic diameter (DH) of about 7.8 nm were produced using gold nanocores having a diameter (d) of about 5.3 nm (TEM), and confirmed to have cancer cell-specific cytotoxicity. On the other hand, mixed-charge nanoparticles having a small-diameter nanocore (about 2.6 nm) showed non-specific cytotoxicity.

Thus, in the present invention, the nanocore may have a diameter (d) of about 4 to 12 nm, as observed with a transmission electron microscope (TEM).

In the present invention, the nanoparticles may have a hydrodynamic diameter (DH) of 6 nm to 20 nm, as measured by dynamic light scattering (DLS).

As used herein, the term “ligand” refers to a material that may be attached to the surface of a nanocore by various interactions such as covalent bonding, non-covalent bonding, coordination bonding, and electrical interaction. The ligand generally affects the physical and chemical properties of the surface of nanoparticles, and is selected and used for various purposes such as electrical properties, crystallographic properties, solubility, stabilization, and functionalization of the nanoparticles. Various ligands for the modification of the nanoparticles have been reported.

In the present invention, the ligand may comprise a nanoparticle anchoring moiety that interacts with the nanoparticles.

As used herein, the term “nanoparticle anchoring moiety” refers to a chemical group in the ligand, which is capable of being attached to the nanoparticles through various interactions. The nanoparticle anchoring moiety may be selected depending on the nature of the nanocore to which the ligand is attached, and an appropriate nanoparticle anchoring moiety according to the central molecule of each nanoparticle is known in the art. For example, nanoparticle anchoring moieties suitable for a metal nanocore include a thiol group, an amine group, a carboxyl group, a phosphine group, a phenol-derived group (or a phenol group), etc., and nanoparticle anchoring moieties suitable for semiconducting quantum dots include a phosphine oxide group, a thiol group, a phosphonyl group, a carboxyl group, etc., and nanoparticle anchoring moieties suitable for transition metal oxides include a carboxyl group, a hydroxyl group, a phosphonyl group, an amine group, etc. (Chem. Rev. 2019, 119, 8, 4819-4880), without being limited thereto.

As used herein, the term “positively charged ligand” refers to a ligand that exhibits a cationic property on the surface of the nanoparticles. Preferably, it may be a ligand that exhibits a cationic property in the presence of a solvent (generally water).

In the present invention, the positively charged ligand may comprise a trimethylamine group or a trimethylammonium group. In trimethylamine or trimethylammonium, the central element N exhibits a cationic property, whereby a monovalent counter-anion such as Cl⁻ or Br⁻ may form an atmosphere in the ligand.

In the present invention, the positively charged ligand may be any one or more selected from among the following Formulas (I) and (II):

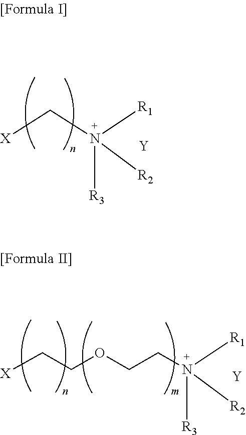

In the present invention, X in Formula I or Formula II above denotes a nanoparticle anchoring moiety.

In the present invention, X in Formula I or Formula II above may be selected from among the following:

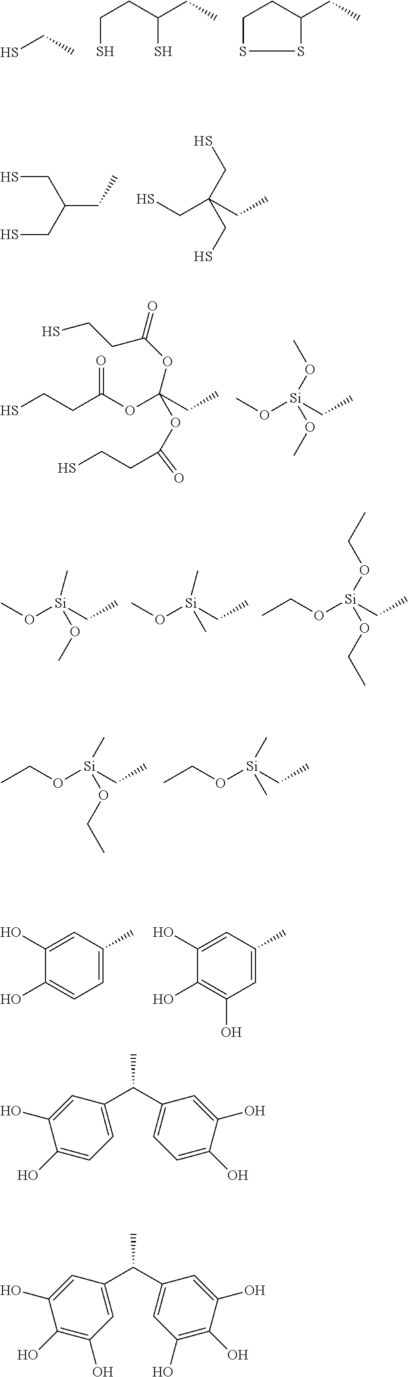

In the present invention, preferably, X in Formula I or Formula II above may be a thiol group or a phenol-derived group.

In the present invention, more preferably, X in Formula I or Formula II above may be a thiol group selected from the group consisting of

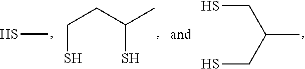

or may be a phenol group selected from the group consisting of

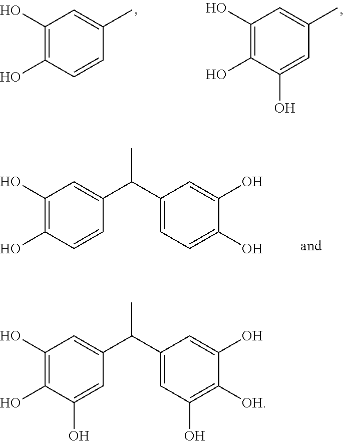

More preferably, X may be

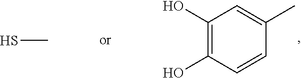

most preferably HS—.

As used herein, the terms “chemical groups,” including “thiol group,” “amine group,” “carboxyl group,” “hydroxyl group,” “phosphonyl group,” and “phosphine group” are used in a broad sense including the chemical groups as well as derivatives derived therefrom.

As used herein, the term “phenol-derived group” is meant to include both a phenol group in which one or more hydroxyl groups are bonded to a benzene ring and derivatives derived therefrom.

In the present invention, Y in Formula I or Formula II denotes a counter ion against N⁺. In the present invention, Y may be a monovalent anion. In the present invention, Y may be a halogen ion, preferably Cl⁻ or Br⁻, most preferably Cl⁻.

In the present invention, R1, R2 and R3 in Formula I or Formula II may be each independently selected from H and CH₃.

In the present invention, n in Formula I or Formula II may be an integer of 1 or more, preferably an integer ranging from 3 to 11, without being limited thereto.

In the present invention, m in Formula I or Formula II may be an integer of 1 or more, preferably an integer ranging from 2 to 6, without being limited thereto.

In the present invention, the positively charged ligand is most preferably TMA used in the Examples.

As used herein, the term “negatively charged ligand” refers to a ligand that exhibits an anionic property on the surface of the nanoparticles. Preferably, it may be a ligand that exhibits an anionic property in the presence of a solvent (generally water).

In the present invention, the negatively charged ligand may comprise a chemical group selected from the group consisting of a carboxyl group, a sulfonate group, and a phosphonate group. The carboxyl group, sulfonate group, and phosphonate group may exhibit an anionic property depending on the surrounding physical and chemical environment.

In particular, when exhibiting an anionic property, a monovalent cation as a counter ion may form an atmosphere in the ligand.

In the present invention, the negatively charged ligand may be any one or more selected from among the following Formulas (III) and (IV):

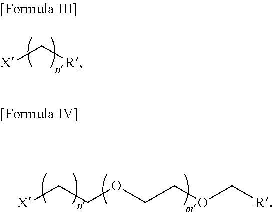

In the present invention, X′ in Formula III or Formula IV denotes a nanoparticle anchoring moiety.

In the present invention, X′ in Formula III or Formula IV may be selected from the group consisting of the following:

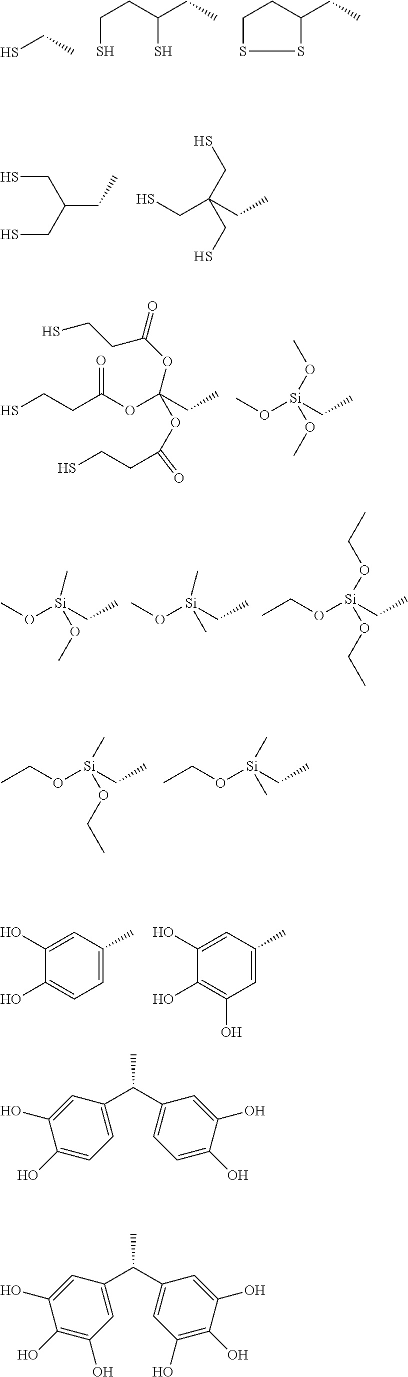

In the present invention, preferably, X′ in Formula III or Formula IV may be a thiol group or a phenol-derived group.

In the present invention, more preferably, X′ in Formula III or Formula IV may be a thiol group selected from the group consisting of

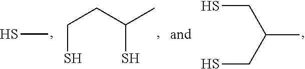

or may be a phenol-derived group selected from the group consisting of

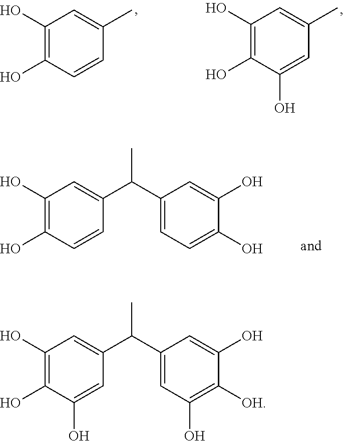

More preferably, X′ may be

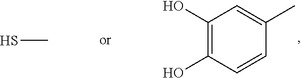

most preferably HS—.

In the present invention, R′ may be a functional group which is as an anionic group.

In the present invention, preferably, R′ may be selected from the group consisting of a carboxyl group, a sulfonate group, and a phosphonate group.

In the present invention, preferably, R′ in Formula III or IV may be selected from the group consisting of

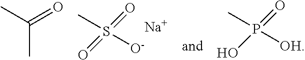

### Na⁺ in

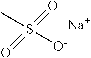

denotes a counter ion. In the present invention, the Na⁺ may be replaced with a monovalent cation.

In the present invention, n′ in Formula III or Formula IV is an integer of 1 or more, preferably an integer ranging from 3 to 11, without being limited thereto.

In the present invention, m′ in Formula III or Formula IV is an integer of 1 or more, preferably an integer ranging from 2 to 6, without being limited thereto.

In one example of the present invention, as the positively charged ligand, positively charged N,N,N-trimethyl(11-mercaptoundecyl) ammonium chloride (TMA) was used, and as the negatively charged ligand, negatively charged 11-mercaptoundecanoic acid (MUA) was used. Mixed-charge nanoparticles were produced by mixing these ligands together in various ratios. Among the produced mixed-charge nanoparticles, the critical ratio between the ligands on the surface of the mixed-charge nanoparticles exhibiting cancer cell-specific cytotoxicity was 61:39 to 91:9 (χTMA: χMUA), and 100:0 (χTMA:χMUA), purely cationic nanoparticles, showed non-specific cytotoxicity as previously reported. In addition, it was confirmed that the zeta potential of mixed-charge nanoparticles with a critical value of 61:39 to 91:9 in water at a pH of 7.4 was in the range of about 10 mV to 35 mV.

Thus, in the present invention, the ratio of the positively charged ligand to the negatively charged ligand on the surface of the mixed-charge nanoparticles may be 51:49 to 99:1 (χ[+]:χ[−]), preferably 55:45 to 98:2 (χ[+]:χ[−]), more preferably about 61:39 to about 91: 9 (χ[+]:χ[−]), even more preferably 70:30 to 85:15 (χ[+]:χ[−]), most preferably about 80:20 (χ[+]:χ[−]), which is a value indicating critical significance for cancer cell-specific cytotoxicity.

In the present invention, depending on the types of positively charged ligand and negatively charged ligand, the ratio of the positively charged ligand to the negatively charged ligand may be appropriately selected and adjusted in order to make the surface net charge positive.

In the present invention, the zeta (ξ) potential of the mixed charge nanoparticles in water at pH 7.4 may be greater than 0 mV since the nanoparticles should exhibit a cationic property. Preferably, the zeta potential in water at pH 7.4 may be 10 mV to 35 mV, which is a critical value. Most preferably, the zeta potential in water at pH 7.4 may be about 15 mV to 25 mV.

The mixed-charge nanoparticles of the present invention may exhibit a pH-dependent aggregation behavior.

In the present invention, the mixed-charge nanoparticles may form aggregates having a size of 10 nm or more at a pH of about 7.5 or less.

In the present invention, the mixed-charge nanoparticles may form aggregates having a size of about 50 to 100 nm at about pH 5.5 to about pH 6.5.

In the present invention, the mixed-charge nanoparticles may form aggregates having a size of about 2,000 nm or more at about pH 4.5 or less.

In the present invention, the mixed-charge nanoparticles may be crystalized at pH 4.5 to pH 5.5 or less.

The mixed-charge nanoparticles of the present invention may be specifically internalized into cancer cells.

The mixed-charge nanoparticles of the present invention may be localized to lysosomes or early endosomes in cancer cells.

The mixed-charge nanoparticles of the present invention may aggregate in multivesicular bodies (MVBs) in cancer cells.

The mixed-charge nanoparticles of the present invention may induce swelling of cancer cell lysosomes, and induce damage to lysosomal membrane integrity due to lysosomal membrane permeability, resulting in lysosomal cell death.

The mixed-charge nanoparticles of the present invention may be accumulated in autolysosomes characterized by multilamellar bodies (MLBs) in normal cells. However, the mixed-charge nanoparticles of the present invention do not form large aggregates in autolysosomes containing MLBs for the following reasons, and thus do not exhibit cytotoxicity:

(1) high pH in MLB-containing organelles (lysosomes, pHL-NORMAL≈4.8; MLB-containing autologous lysosomes, pHMLB <6.1);

(2) Physical presence of neutral lipids in MLBs42;

(3) Maintenance of a functional and dynamic lysosomal pool by the transient nature of lysosomal fusion events.

In the present invention, when the mixed-charge nanoparticles are internalized into normal cells, they may be released out of the cells.

In one example of the present invention, cancer cell-specific cytotoxicity of the produced mixed-charge nanoparticles against a total of 13 cancer cell lines (sarcoma, melanoma, breast cancer, prostate cancer and lung cancer) and 4 normal cell lines (non-cancer cells, including human fibroblasts) was examined.

In another example of the present invention, it was demonstrated that the cancer cell-specific cytotoxicity of the mixed-charge nanoparticles of the present invention does not result from simply diluting the negatively charged ligand with the positively charged ligand, but results from formation of large aggregates by pH-dependent aggregation of the nanoparticles and crystallization of the nanoparticles, which occur specifically in cancer cell lysosomes.

Considering the cancer cell-specific cytotoxic mechanism of mixed-charge nanoparticles confirmed in the examples of the present invention and that most tumor cells including solid cancer and blood cancer create a lower pH environment than normal cells, it is obvious that the mixed-charge nanoparticles may be used to induce selective death of not only sarcoma, melanoma, breast cancer, prostate cancer and lung cancer used in the examples of the present invention, as well as various tumors.

Therefore, in another aspect, the present invention is directed to a composition for cancer cell death containing the mixed-charge nanoparticles.

In still another aspect, the present invention is directed to a method for inducing cancer cell death comprising a step of treating a subject with the mixed-charge nanoparticles or administering the mixed-charge nanoparticles to a subject.

In yet another aspect, the present invention is directed to the use of the mixed-charge nanoparticles for inducing cancer cell death.

In still yet another aspect, the present invention is directed to the use of the mixed-charge nanoparticles for preparing a composition for inducing cancer cell death.

In the present invention, the composition for inducing cancer cell death may be used for complete/partial death of cancer cells, or for reduction in the volume of cancer tissue, and may be used in various environments and under various conditions regardless of in vitro, in vivo, or ex vivo.

In the present invention, the composition for inducing cancer cell death may further contain a suitable solvent, carrier, excipient, etc. for containing the mixed charge nanoparticles.

In the present invention, the composition for inducing cancer cell death may contain, in addition to the mixed-charge nanoparticles, a second active ingredient exhibiting an effect of inhibiting cancer cell proliferation or killing cancer cells.

As used herein, the term “subject” may include an animal suffering from cancer, more preferably a mammal, and most preferably a human, and also includes organs, tissues, and cells containing cancer cells isolated from the animal.

Therefore, in another aspect, the present invention is directed to a pharmaceutical composition for preventing or treating cancer containing the mixed-charge nanoparticles.

In still another aspect, the present invention is directed to a method for preventing or treating cancer containing a step of treating a subject with the mixed charge nanoparticles or administering the mixed charge nanoparticles to a subject.

In yet another aspect, the present invention is directed to the use of the mixed charge nanoparticles for prevention or treatment of cancer.

In still yet another aspect, the present invention is directed to the use of the mixed-charge nanoparticles for preparing a pharmaceutical composition for prevention or treatment of cancer.

As used herein, the term “cancer” refers to a proliferative disease characterized by uncontrolled growth or proliferation of cells. Cancer cells may spread to other parts of the body either locally or through the bloodstream and lymphatic system. In addition to the examples of cancer described herein, various examples of cancer are known in the art. In the present invention, the term “cancer” is used interchangeably with the term “tumor,” and is meant to include not only carcinoma in situ, metastatic cancer, solid cancer, non-solid cancer, malignant cancer and malignant tumors, but also premalignant cancer.

In the present invention, examples of the cancer or tumor include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, etc.

In the present invention, the cancer may have lysosomes having an acidity of preferably about pH 4.5 to pH 5.5.

In the present invention, the cancer may preferably be a solid cancer. More preferably, the cancer may be sarcoma, melanoma, breast cancer, prostate cancer, or lung cancer, as confirmed in one example of the present invention. More specifically, the cancer may be fibrosarcoma, human breast adenocarcinoma, melanoma, prostate carcinoma, mammary duct carcinoma, or lung cancer.

Since the mixed-charge nanoparticles of the present invention induce cancer cell death by cancer-selective cytotoxicity, they exhibit anti-tumor effects. As used herein, “anti-tumor effects” refers to a decrease in tumor volume, a decrease in the number of tumor cells, a decrease in tumor cell proliferation, a decrease in the number of metastases, an increase in overall or progression-free survival, an increase in life expectancy, or alleviation of various physiological symptoms associated with tumors.

As used herein, the term “prevention” refers to any action of inhibiting, or delaying the onset of, a disease of interest, specifically cancer, by administration of the pharmaceutical composition according to the present invention.

As used herein, the term “treatment” refers to any action that alleviates or beneficially changes symptoms of a disease of interest by administration of the pharmaceutical composition according to the present invention.

The pharmaceutical composition may further contain suitable carriers, excipients and diluents, which are commonly used in pharmaceutical compositions, in addition to the mixed-charge nanoparticles of the present invention as an active ingredient.

Carriers, excipients and diluents that may be contained in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. For formulation, the composition is usually prepared using diluents or excipients such as a filler, an extender, a binder, a wetting agent, a disintegrant, and a surfactant.

The pharmaceutical composition according to the present invention may be formulated in various dosage forms according to a conventional method. Suitable dosage forms include, but are not limited to, oral dosage forms such as tablets, pills, powders, granules, dragées, hard or soft capsules, solutions, suspensions or emulsions, injections, aerosols, external preparations, suppositories, and sterile injection solutions.

The pharmaceutical composition according to the present invention may be prepared in a suitable dosage form using a pharmaceutically inert organic or inorganic carrier. That is, when the dosage form is a tablet, a coated tablet, a dragée, or a hard capsule, it may contain lactose, sucrose, starch or a derivative thereof, talc, calcium carbonate, gelatin, and stearic acid or a salt thereof. In addition, when the dosage form is a soft capsule, it may contain vegetable oil, wax, fat, and a semi-solid and liquid polyol. In addition, when the dosage form is a solution or syrup, it may contain water, polyol, glycerol, and vegetable oil.

The pharmaceutical composition according to the present invention may further contain a preservative, a stabilizing agent, a wetting agent, an emulsifying agent, a solubilizing agent, a sweetening agent, a coloring agent, an osmotic pressure adjusting agent, an antioxidant, and the like, in addition to the carrier described above.

The pharmaceutical composition according to the present invention may be administered in a pharmaceutically effective amount. As used herein, the term “pharmaceutically effective amount” refers to an amount sufficient to induce or increase an immune response and not cause side effects or serious or excessive immune response. A suitable dosage may be determined in various ways depending on factors such as formulation method, administration method, the patient's age, weight, sex, pathological condition and diet, administration time, administration route, excretion rate, and response sensitivity. Various general considerations in determining a “pharmaceutically effective amount” are known to those of skill in the art, and are described, for example, in Gilman et al., eds., Goodman and Gilman's: The Pharmacological Bases of Therapeutics, 8^{th} ed., Pergamon Press, 1990, and Remington's Pharmaceutical Sciences, 17^{th} ed., Mack Publishing Co., Easton, Pa., 1990. The pharmaceutical composition according to the present invention may be administered individually or in combination with other therapeutic agents, may be administered sequentially or simultaneously with a conventional therapeutic agent, and may be administered in a single or multiple dosage form. It is important to administer the pharmaceutical composition in the minimum amount that may exhibit the maximum effect without causing side effects, in view of all the above-described factors, and this amount may be easily determined by a person skilled in the art.

The pharmaceutical composition of the present invention may be administered to an individual by various routes. The pharmaceutical composition may be administered orally or parenterally. For parenteral administration, the pharmaceutical composition may be administered by intravenous injection, subcutaneous injection, intramuscular injection, intraperitoneal injection, endothelial administration, topical administration, intranasal administration, intrapulmonary administration, or intrarectal administration. However, because a protein or a peptide is digested when administered orally, the active ingredient in the composition for oral administration should may be coated or formulated so as to be protected from degradation in the stomach. In addition, the pharmaceutical composition may be administered by any device by which the active ingredient may be delivered to target cells.

The mode of administration of the pharmaceutical composition according to the present invention may be easily selected depending on the dosage form, and the pharmaceutical composition may be administered orally or parenterally. Dosage may vary depending on the patient's age, sex and weight, severity of disease, and route of administration.

In the pharmaceutical composition, the term “subject” or “individual” as used herein means a subject suffering from cancer, and includes any living organism suffering from cancer, preferably an animal, more preferably a mammal, most preferably a human, without being limited thereto.

### EXAMPLES

Hereinafter, the present invention will be described in more detail with reference to examples. These examples are only for illustrating the present invention, and it will be apparent to those of ordinary skill in the art that the scope of the present invention is not to be construed as being limited by these examples.

### Example 1: Production and Characterization of Mixed-Charge Nanoparticles (MCNPs)

### Example 1-1: Design and Production of Mixed-Charge Nanoparticles

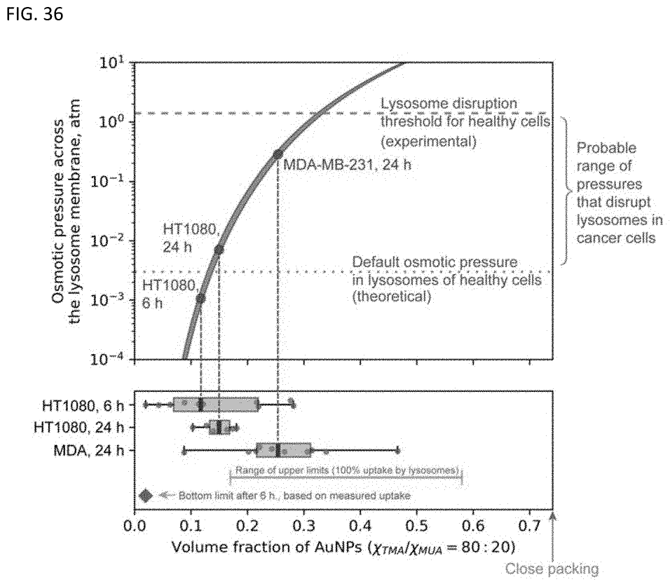

The size of the mixed charge nanoparticles was determined considering the following considerations. For nanoparticles with a hydrodynamic diameter (DH) greater than about 100 nm, they cannot efficiently penetrate into tissues or cells. Gold particles of several tens of nm have a van der Waals force (vdW) that overwhelms the electrostatic interaction. In addition, nanoparticles with a DH of less than about 6 nm can penetrate rapidly into the extravascular space, but are easily cleared from the circulatory system by the kidneys, and thus have a very short in vivo half-life. Especially, nanoparticles having gold nanocores with d<2.5 nm may exhibit non-selective cytotoxicity (see FIG. 8). Therefore, the present inventors designed mixed-charge nanoparticles using gold nanocores having a diameter of about 4 to about 12 nm, as observed with a transmission electron microscope, in order to ensure that the mixed-charge nanoparticles have an appropriate hydrodynamic diameter.

Mixed charge nanoparticles were synthesized by the method disclosed previously by the present inventors (J. Am. Chem. Soc. 135, 6392-6395 (2013); Angew. Chem. Int. Ed. 55, 8610-8614 (2016)). First, in order to reduce Au³⁺ ions, a toluene solution (3 mL) containing 58 mg of tetrabutylammonium borohydride (TBAB) and 111 mg of dilauryl dimethylammonium bromide (DDAB) was injected into a toluene solution (7 mL) containing 222 mg of dodecyl amine (DDA), 277 mg of DDAB and 24 mg of HAuCl₄·3H₂O. The mixed solution was aged by stirring in the dark overnight to obtain a solution of DDA-protected gold nanoparticle cores having a diameter of about 2 to 4 nm. A growth solution of DDA (2.6 g), DDAB (1 g) and HAuCl₄·3H₂O (224 mg) in toluene (60 mL) was prepared and then added to the nanoparticle core solution with stirring. Next, a toluene solution (22 mL) of DDAB (1.5 g) and hydrazine monohydrate (145 mg/220 μL) was added dropwise to the nanoparticle solution. The resulting Au-DDA nanoparticle solution was aged by stirring in the dark for 24 hours.

To remove excess DDA, 0.13 mmol of DDA-capped nanoparticles (NPs) were quenched with methanol (50 mL) and allowed to precipitate for 3 to 4 hours. Solvent was then decanted, the precipitate was dissolved in toluene (20 mL) and the mixture of TMA and MUA thiols in dichloromethane (10 mL) was added to initiate the ligand exchange reaction. TMA is always positively charged Regardless of pH, and MUA is sensitive to pH. MUA is deprotonated at pH 7.4 and shows a negative charge, but is neutral at acidic pH.

The total amount of MUA and TMA thiols added was 0.13 mmol and the molar ratios of thiols in solution were 100:0, 89:11, 75:25, 50:50, 40:60, 33:67, 25:75 and 0:100 (TMA:MUA). Because the ratios of ligands used in the solution were not the same as the resulting on-particle ligand ratios, the exact composition of thiols on the surface of the produced MCNPs was analyzed by core-etching/NMR analyses (J. Am. Chem. Soc. 135, 6392-6395 (2013)) and by electrostatic titration (electrostatic titration, J. Phys. Chem. C 120, 4139-4144 (2016)). The nanoparticle solutions were incubated for about 18 hours in the dark. Solvent was subsequently decanted and precipitates were washed with dichloromethane (3×50 mL) and acetone (30 mL). Finally, acetone was decanted and the precipitates were dried naturally. MUA ligands were deprotonated by adding TMAOH, and the precipitates were dissolved in water (10 ml) to yield an ˜about 10 mM (in terms of Au3+ ions)/2 μM (in terms on nanoparticles) stock solution of NPs at a pH of about 11.

For “charge-diluted” 80:C9 nanoparticles, the mixture of TMA and nonanethiol ligands with a molar ratio of the two thiols in solution of 75:25 was used in the ligand exchange reaction. The gold core diameters of the produced MCNPs determined from TEM images taken with a JEM-2100 system (JEOL, USA) and analyzed using NIS-Elements software (Nikon, Japan) were d=5.3±0.7 nm.

It was confirmed that the hydrodynamic diameter (DH) determined by dynamic light scattering (DLS) in 1730 backscatter mode was about 7.8±0.4 nm. The ratios between the ligands (χTMA:χMUA) on the surfaces ratio of the produced nanoparticles were 100:0, 91:9, 80:20, 73:27, 61:39, 40:60, 12:88 and 0:100, respectively.

100:0 or 0:100 nanoparticles were selected as a control representative of nanoparticles having a positive or negative net charge, respectively.

### Example 1-2: pH-Dependent Aggregation Properties of MCNPs

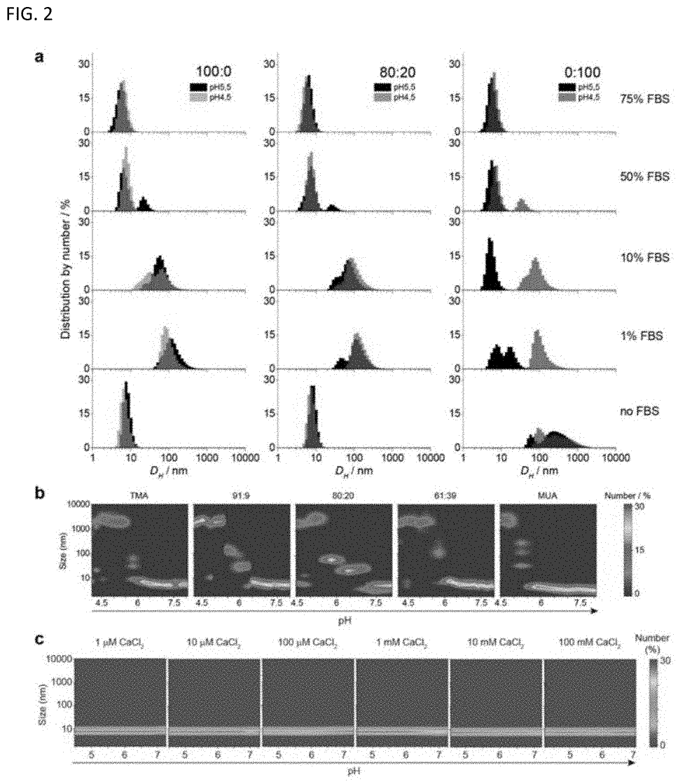

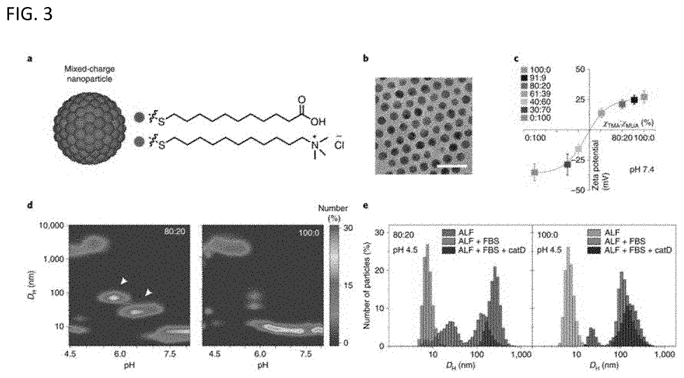

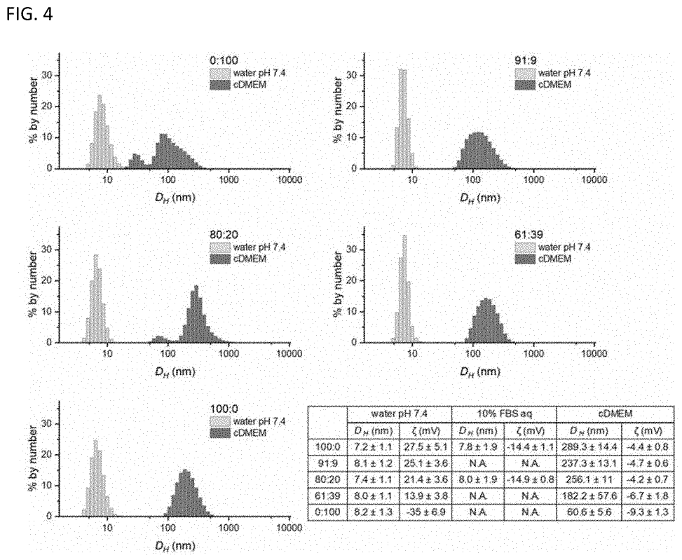

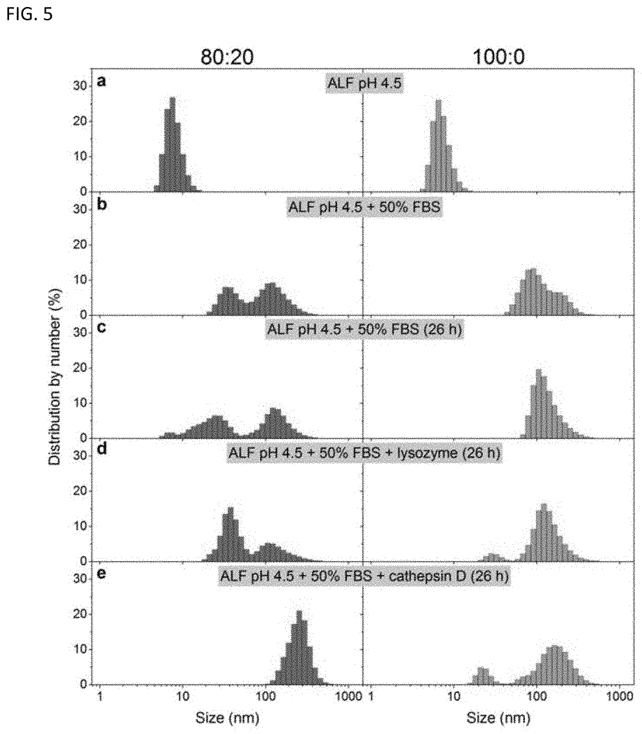

To confirm the aggregation properties of MCNPs, 10 to 15 μL of 5 mM HCl was added to a 50 nM nanoparticle solution and titrated in a stirred vial. After addition, the solution was allowed to equilibrate for about 45 minutes, and the size distribution of nanoparticles was examined immediately after pH measurement. The produced MCNPs exhibited pH-dependent aggregation. In particular, it was confirmed that, as the pH decreased, the nanoparticles formed larger aggregates (FIG. 2b). It was shown that, in water supplemented with 10% FBS, all types of MCNPs did not aggregate at pH 7.4, but gradually aggregated as the pH decreased, and aggregated into clusters having a diameter of about 50 to about 100 nm at about pH 5.5 to pH 6.5 (FIG. 3d). In addition, it was confirmed that super-particles of about 2 μm were formed at a pH of 5.5 or less (FIGS. 2 and 4).

Unlike all types of nanoparticles (χTMA:χMUA=100:0, 91:9, 80:20, 61:39 and 0:100), which showed a common tendency to show larger aggregates with decreasing pH, it was confirmed that the pH at which aggregation starts and the number of moderate aggregates depended on the charge balance of the nanoparticle surface (FIG. 2b). In particular, MUA-functionalized nanoparticles were stable up to pH 5.5, whereas 80:20 MCNPs started to aggregate from about pH 7 (FIG. 3d). In addition, when CaCl₂ was added at various concentrations (1 μM, 10 μM, 100 μM, 1 mM, 10 mM or 100 mM), it remained non-aggregated over the entire pH range and ionic strength spectrum (FIG. 2c).

Considering the pH-dependent aggregation properties of these MCNPs, clusters of about 50 to about 100 nm formed under extracellular pH conditions are capable of cellular uptake through endocytosis (Chem. Rev. 114, 1258-1288 (2014)), and larger diameter supraparticles can be formed at low pH conditions in endosomes or lysosomes.

### Example 1-3: Aggregation Behavior of MCNPs in Whole Cell Culture Medium

To understand the aggregation behavior of MCNPs in cell culture media, MCNPs were mixed with cDMEM (DMEM containing 10% FBS, 1 mM sodium pyruvate and 25 μg/mL gentamicin), then the Cree and zeta potentials were determined and compared with the results obtained in water at pH 7.4. As shown in FIG. 4, it was confirmed that, when mixed with cDMEM, the hydrodynamic diameter (DH) of MCNPs increased from about 7-8 nm, which is the diameter in water, up to about 250 nm. At the same time, the zeta potential of aggregates for all types of MCNPs changed from positive values to negative values (FIG. 4). When the cationic particles and the cell culture medium were mixed together, the zeta potential showed a weak negative value, which is the same as previously reported in the literature (Nano Lett. 11, 772-780 (2011); PLoS One 12, e0169552 (2017)). This change is due to the formation of a protein corona around the nanoparticles and/or their aggregates.

### Example 1-4: Aggregation Properties of MCNPs in Lysosome-Mimicking Solution

To assess the aggregation behavior of MCNPs in a lysosomal environment, aggregation of MCNPs coated with serum proteins was assessed in artificial lysosomal fluid (ALF, pH 4.5). ALF, a high ionic strength buffer, is very similar to the intracellular lysosomal environment in terms of pH (acidity), ionic strength ([Cl]=50 mM, [Ca²⁺]=0.8 mM, [Na⁺]=205 mM) and many other inorganic and organic constituents, but does not contain proteolytic enzymes (ACS Nano 6, 1513-1521 (2012). ALF at pH 4.5 and ALF at pH 5.5 adjusted by adding 2M NaOH were used as a lysosomal-like environment. MCNPs having 80:20 (TMA:MUA) surface ligands formed clusters with a size of 100 nm in the presence of FBS, and formed aggregates of about 260 nm when the lysosomal protease cathepsin D was added. On the other hand, purely cationic nanoparticles with 100:0 surface ligands showed only slight growth or a decrease in cluster diameter (FIGS. 2a, 3e and 5). On the other hand, 0:100 pure MUA nanoparticles showed rapid aggregation, which is believed to be because the electrical repulsion of the head group was weakened by protonation of the MUA ligand at acidic pH.

In addition, to reflect the protein-rich environment of lysosomes, ALF was supplemented with the serum protein FBS. FBS was believed to be lower than the expected protein concentration inside lysosomes, but only supplementation of FBS inhibited the aggregation of nanoparticles (FIG. 2a). From the above results, it is believed that serum proteins exhibiting a negative charge are attracted to nanoparticles exhibiting a positive net charge mainly through an electrostatic reaction, and pure MUA nanoparticles exhibiting a neutral charge at the test pH are attracted through other interactions such as hydrogen bonding. The formation of a protein corona due to the adsorption of these serum proteins to the nanoparticles can cause the nanoparticles to be dispersed without aggregation.

On the other hand, the aggregation of MCNPs in ALF was shown to be rather promoted at an intermediate concentration of serum protein (about 1 to 10% FBS) (FIG. 2a). This is similar to an environment in which the protein corona of nanoparticles is digested by protease in the lysosome after being introduced into the cell. In order to more specifically assess the aggregation behavior following protein corona digestion, 50 nM nanoparticles were incubated in 50% FBS solution in ALF for 1 hour at 37° C., and then centrifuged at 9391×g to remove excess protein, and the pellet was re-dispersed in fresh ALF to obtain aggregates of small size. These aggregates were incubated without any treatment (control, FIG. 5c) or in the presence of lysozyme (control, non-lysosomal glycoside hydrolase, final concentration 3 μg/mL, FIG. 5d) or cathepsin D (lysosomal protease, final concentration 3 μg/mL, FIG. S3e) at 37° C. for 26 hours. Digestion of protein corona by cathepsin D significantly increased the aggregate size of 80:20 MCNPs. On the other hand, the aggregate size of the 100:0 nanoparticles rather decreased, which is believed to be due to the electrostatic repulsive force between the surfaces of the purely cationic nanoparticles (FIG. 3e).

The results of this in vivo environment-mimicking test suggest that the aggregation properties of MCNPs in vivo do not depend on the ionic strength of the environment, but may depend on degradation of the protein corona by lysosomal proteases in a low-pH environment.

### Example 2: The Ability of Mixed-Charge Nanoparticles to Selectively Kill Cancer Cells

Cytotoxicity studies were performed on the following two sets of cancer and normal cells:

i) HT1080 fibrosarcoma vs. mouse embryonic fibroblasts (MEF)

ii) MDA-MB-231 breast adenocarcinoma vs. MCF-10A

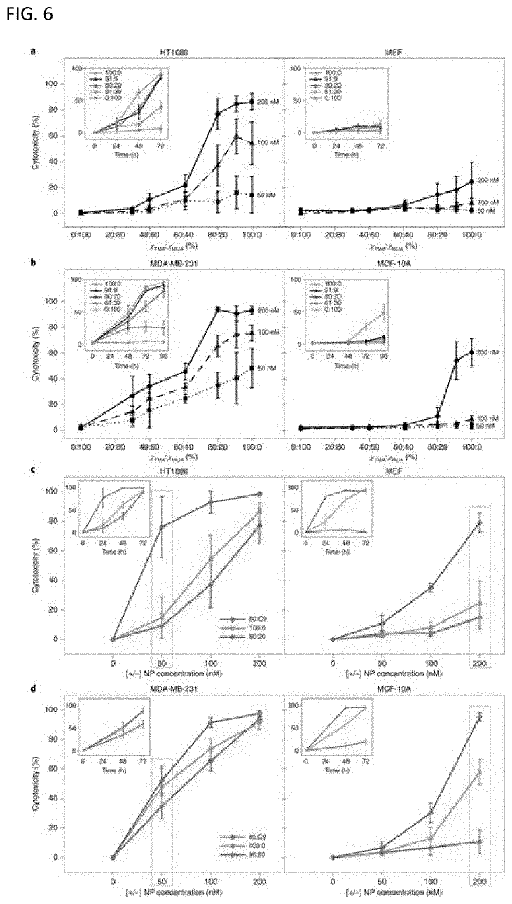

As shown in FIGS. 6a and 6b, it was confirmed that, when the cells were continuously exposed to MCNPs, cancer cells showed cell death in a dose- and time-dependent manner, whereas cytotoxicity to normal cells was very insignificant. In particular, it was shown that the effect of the nanoparticles on cancer cell death depends on the ratio between ligands (χTMA:χMUA) on the surface monolayer of the nanoparticles.

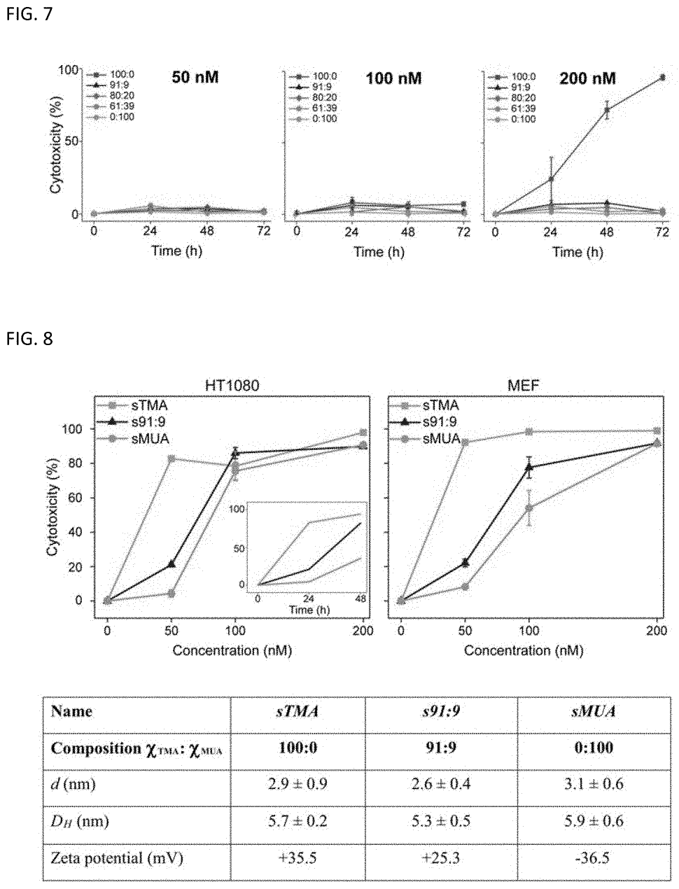

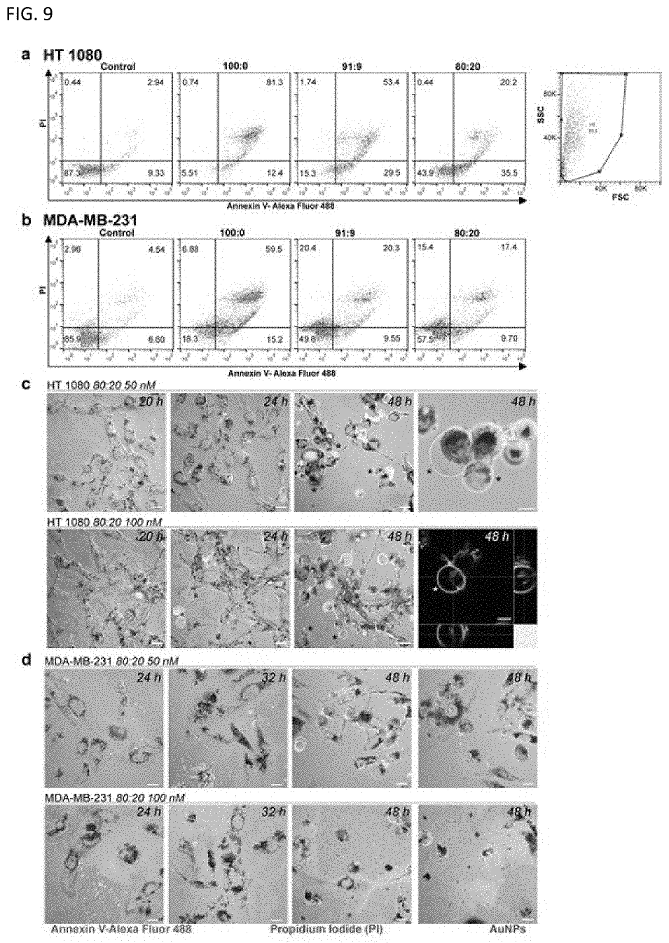

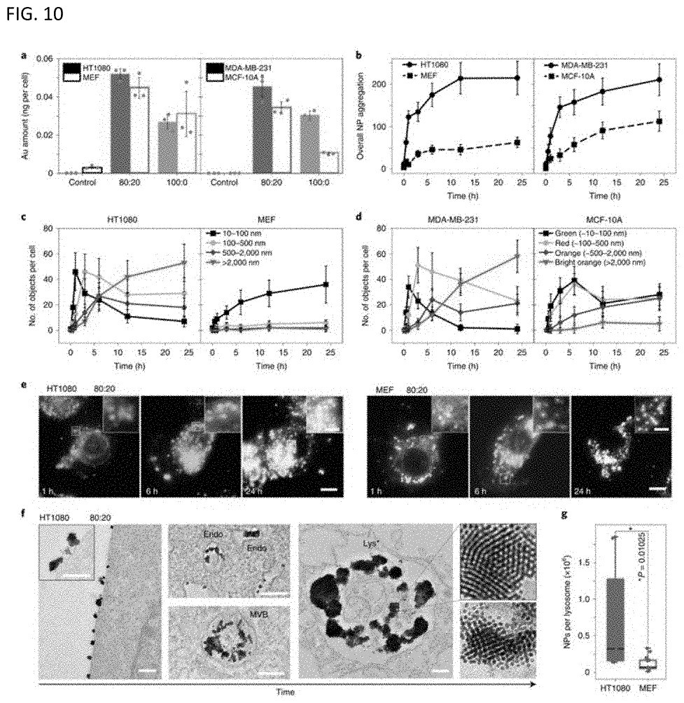

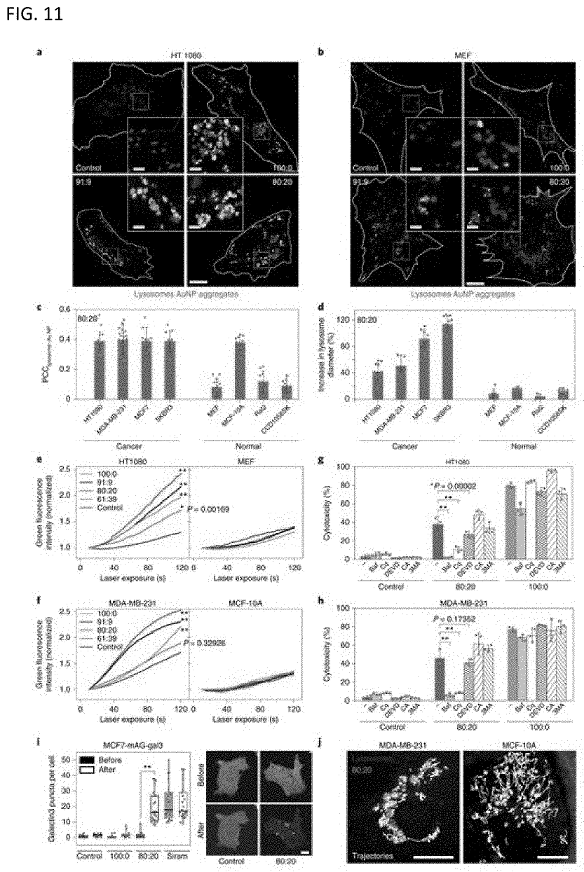

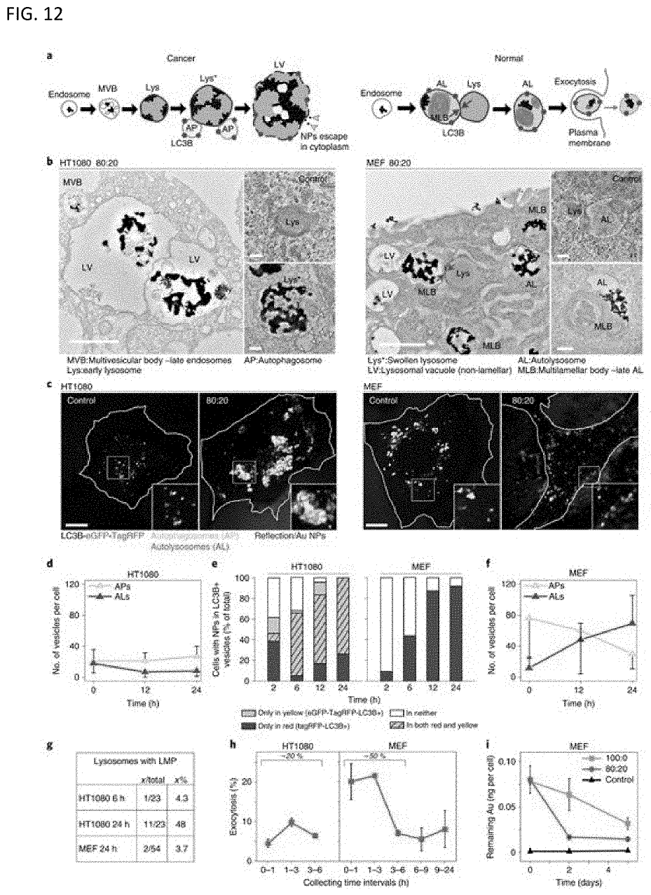

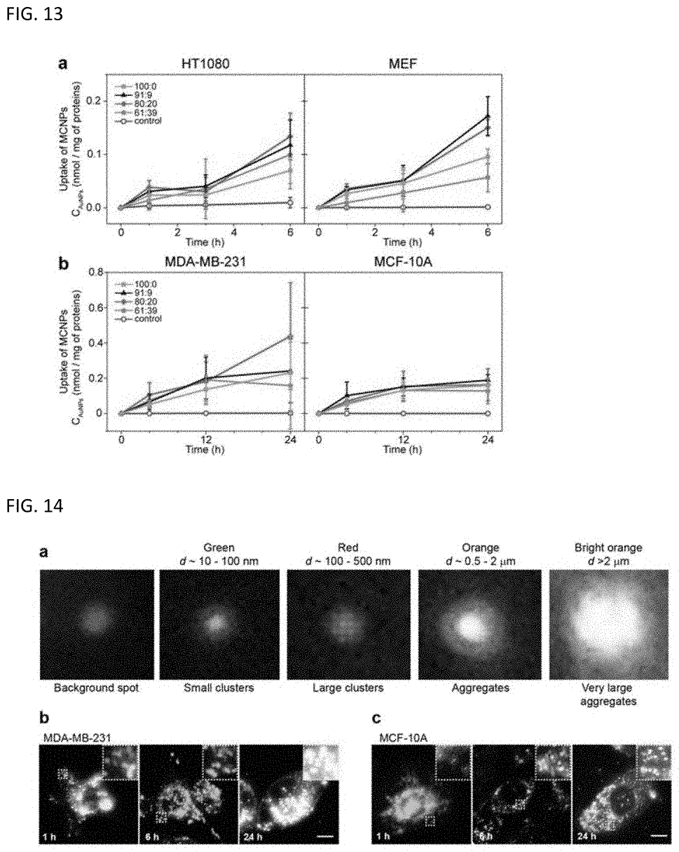

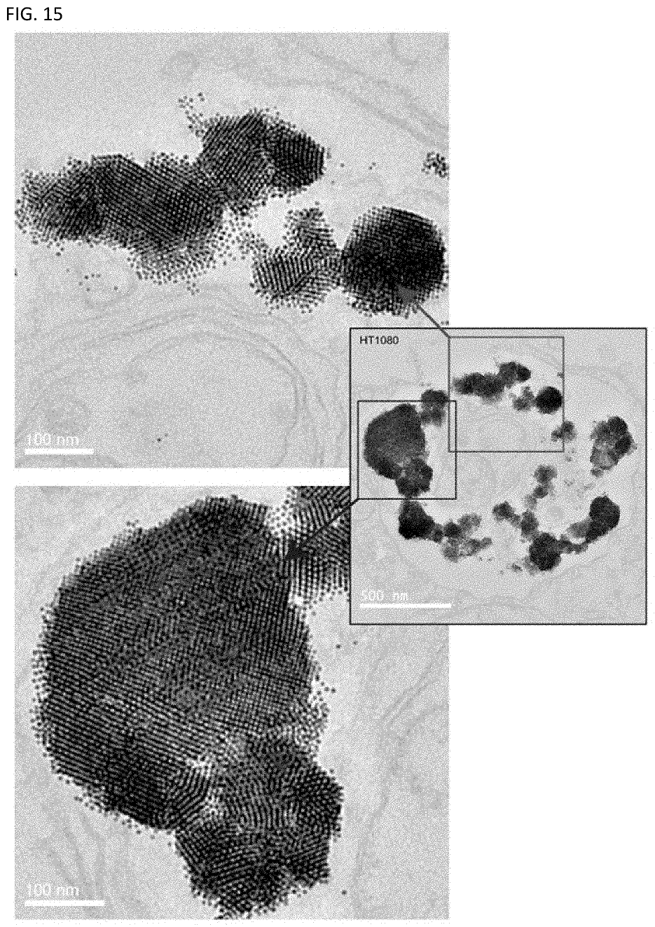

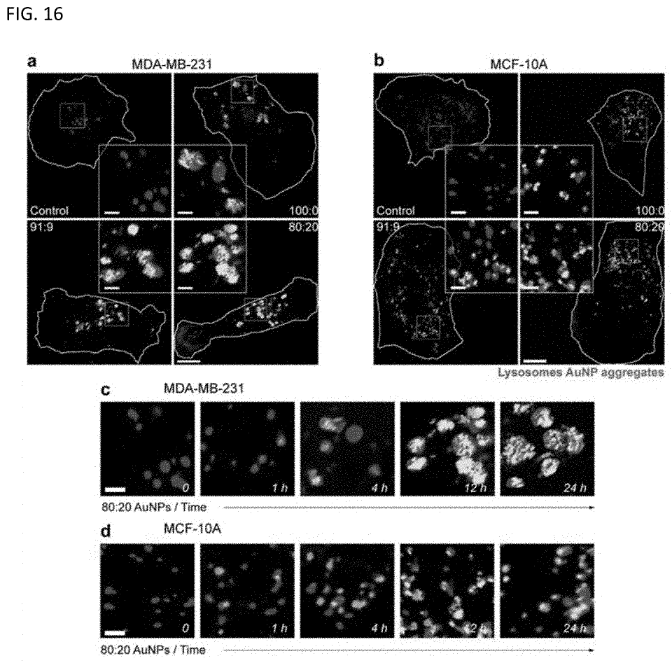

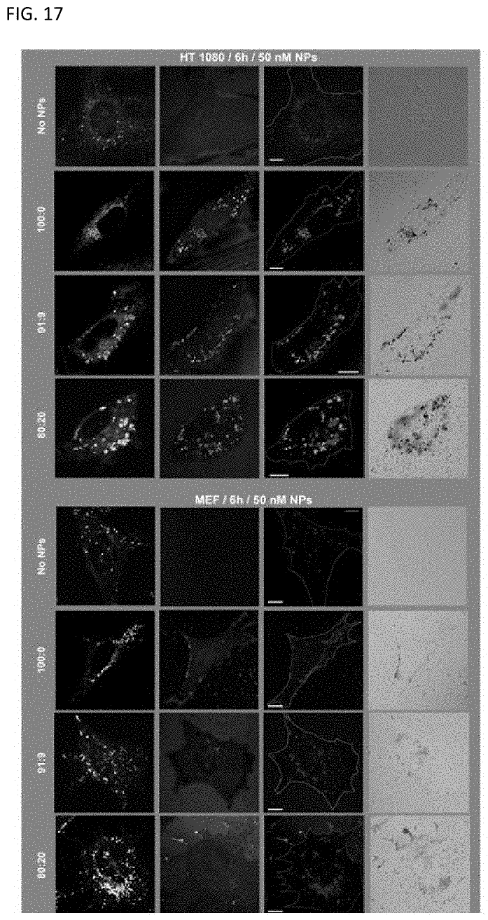

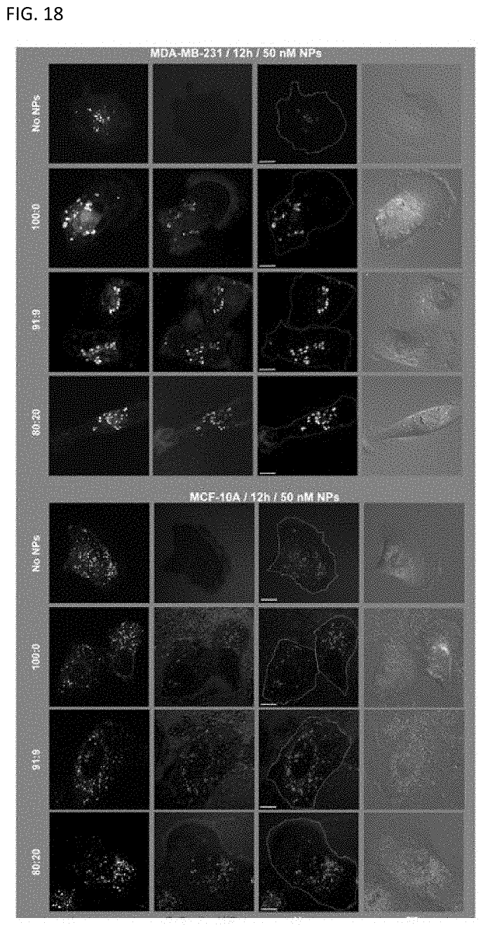

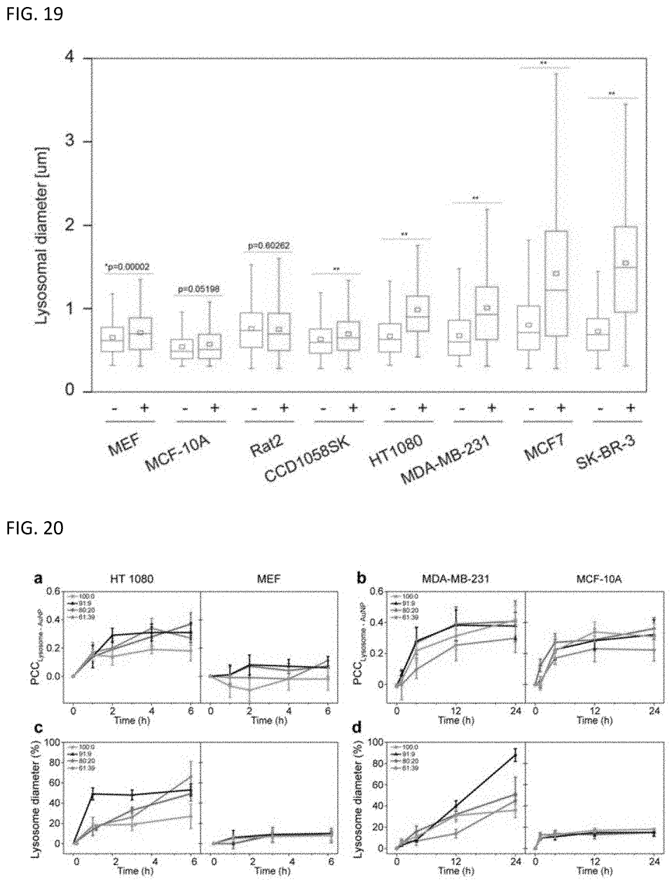

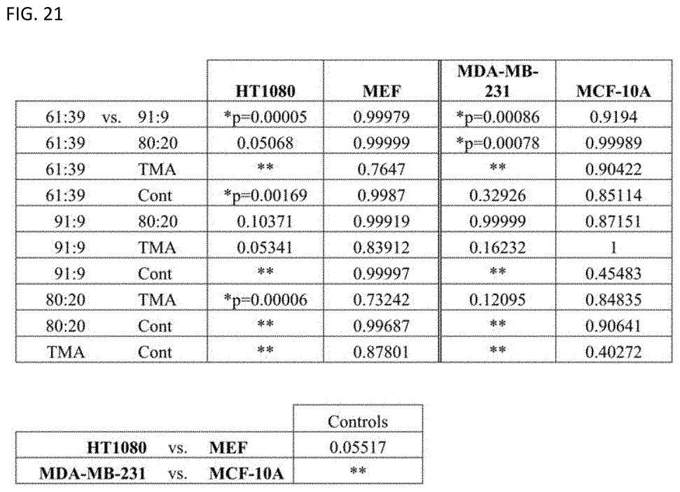

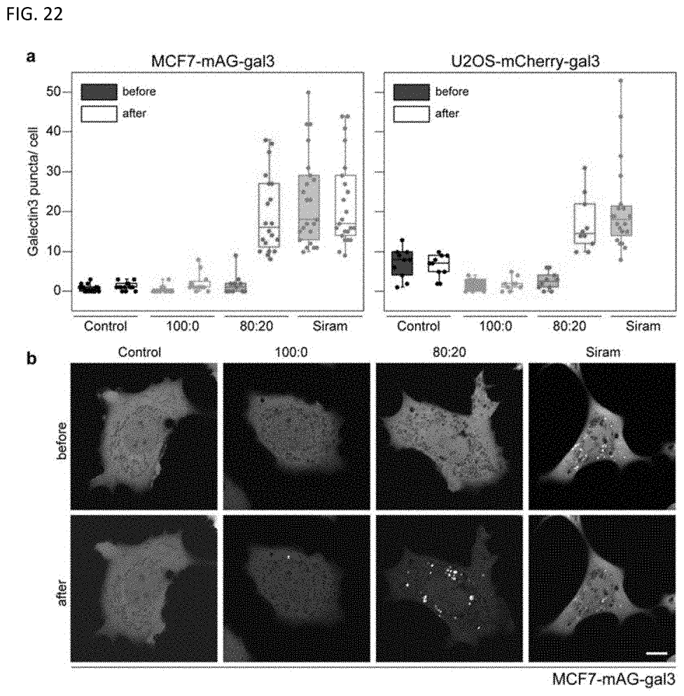

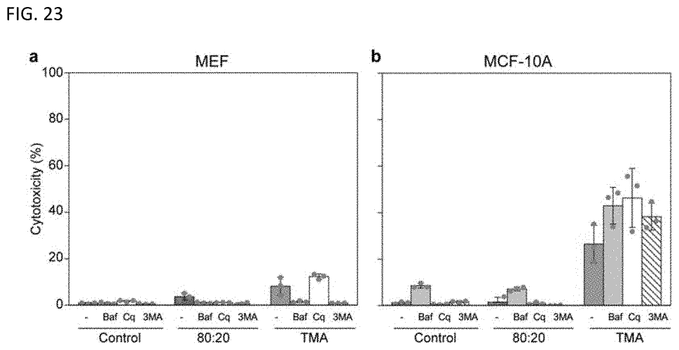

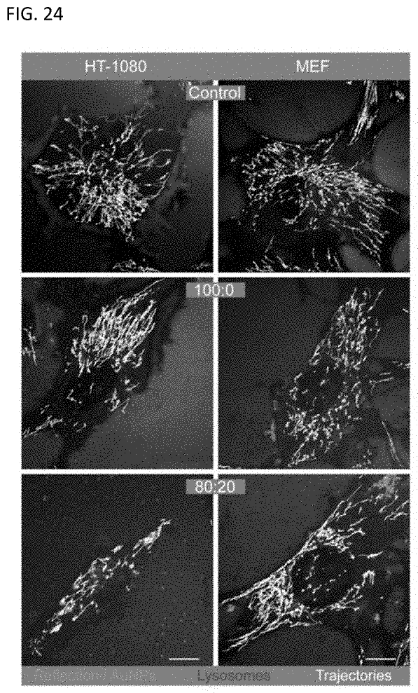

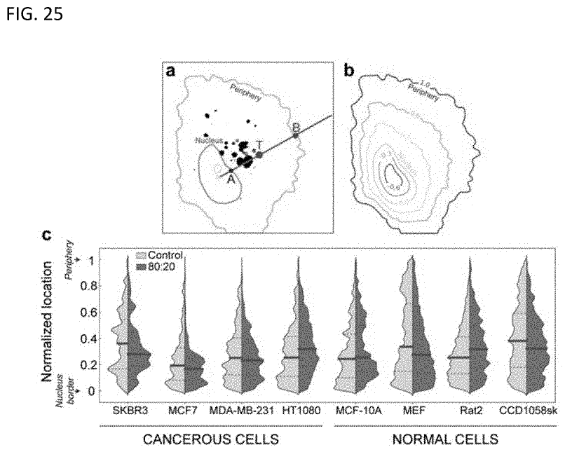

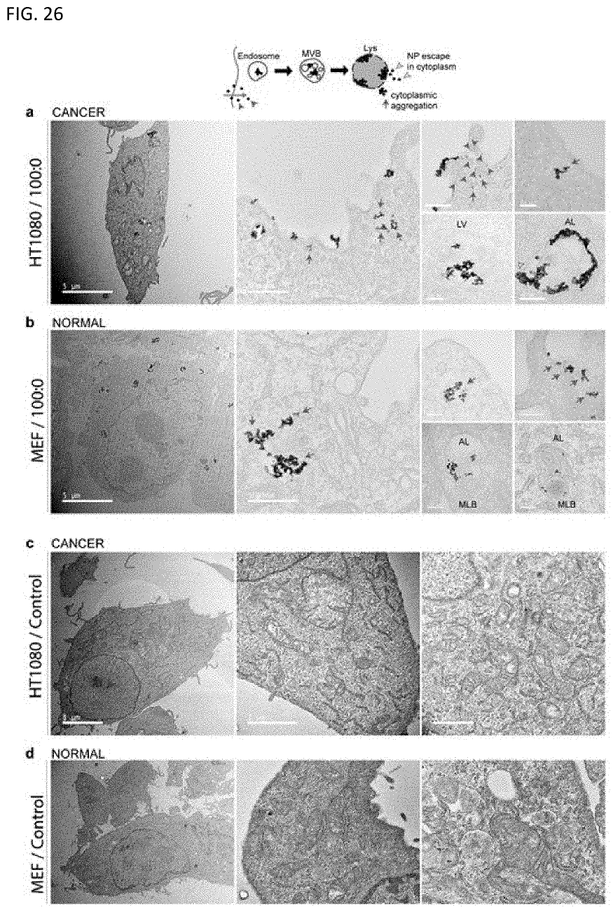

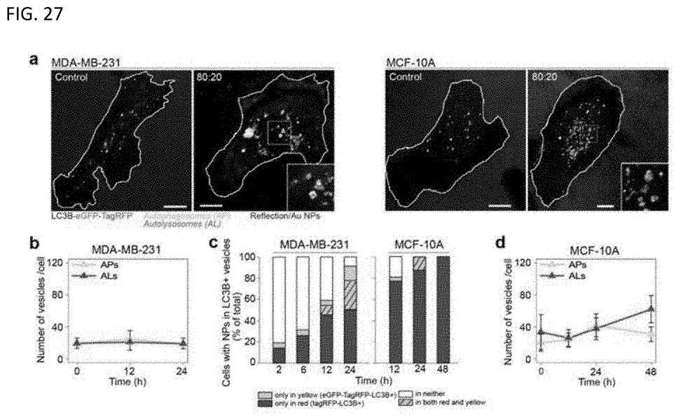

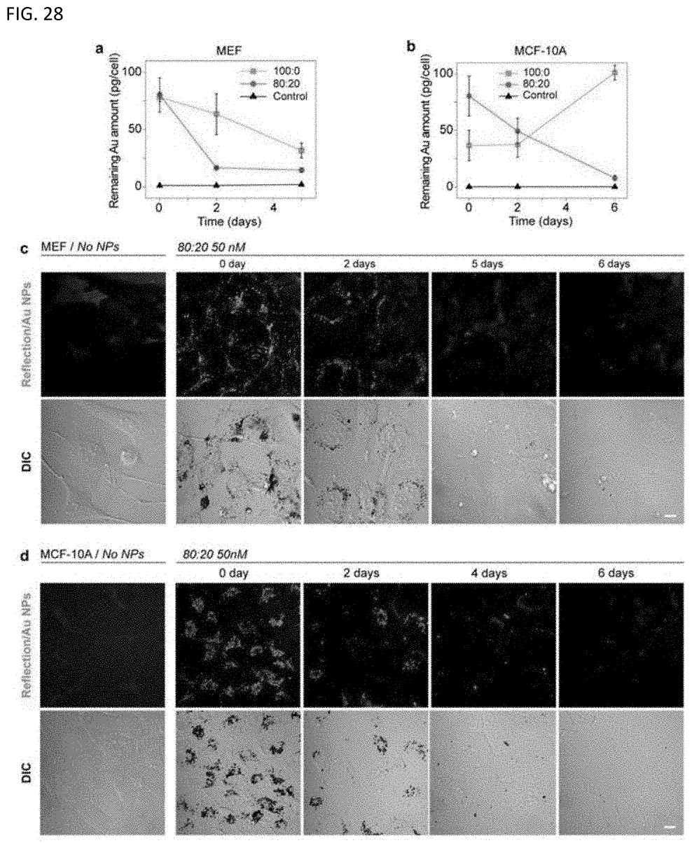

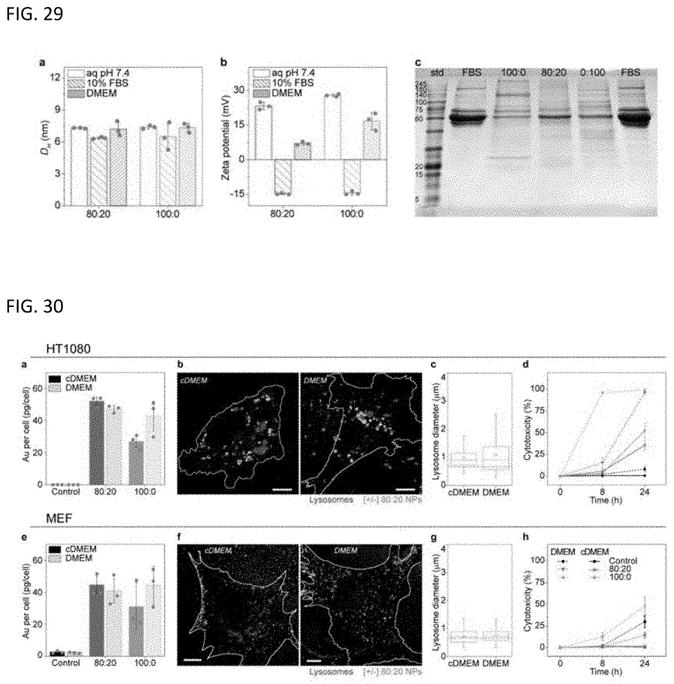

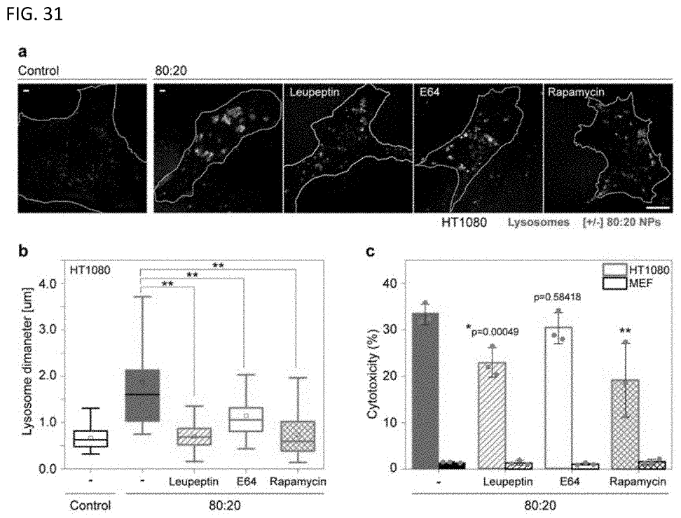

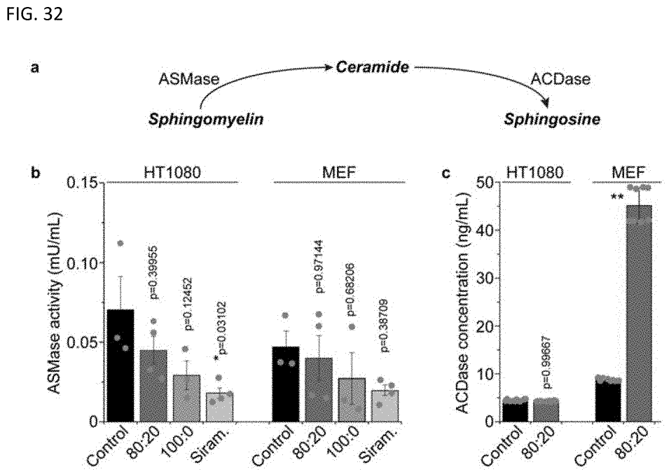

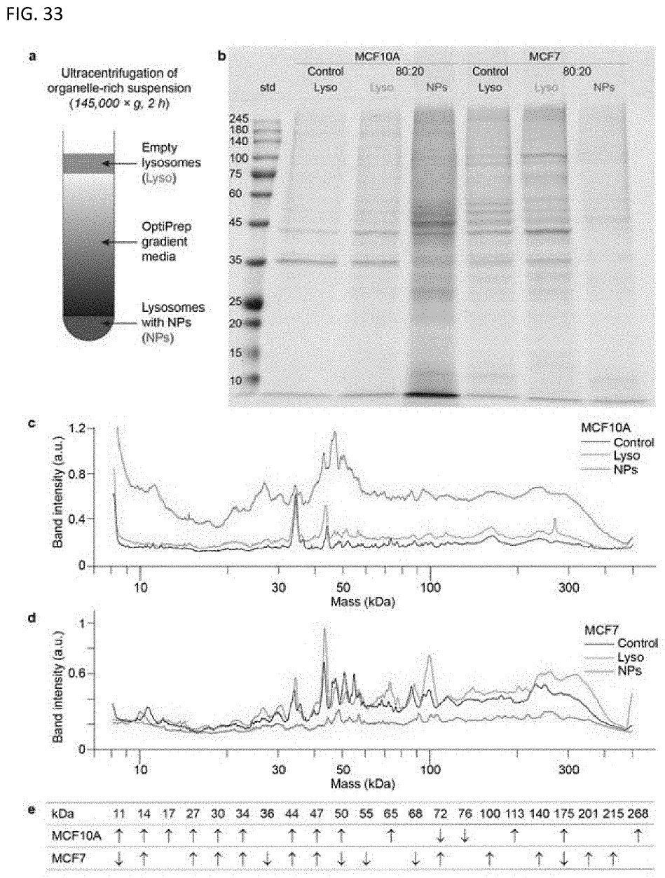

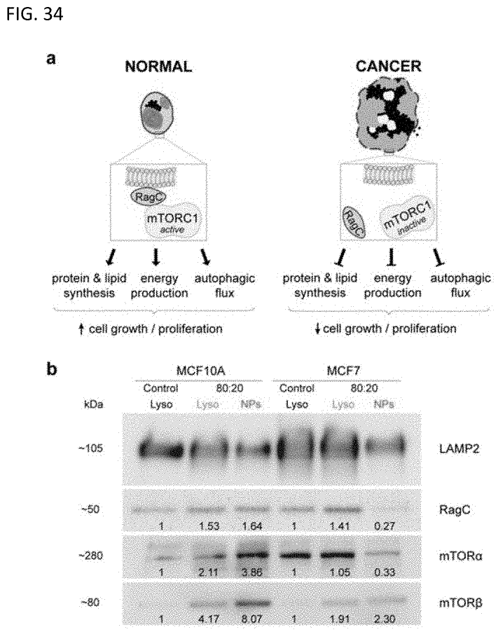

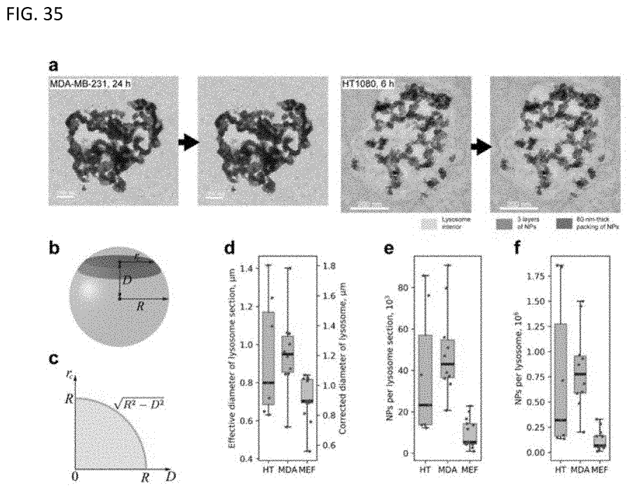

Long-term application studies of the nanoparticles also revealed the cancer cell-selective cytotoxicity of MCNPs. In particular, MCNPs with [χTMA:χMUA]=80:20 did not show toxicity to normal cells over a very long exposure time (72 hours, 200 nM). On the other hand, pure TMA nanoparticles (χTMA:χMUA=100:0), which are the same cationic net charged nanoparticles, showed high toxicity even to normal cells (FIGS. 6c, 6d and 7). This trend can be confirmed by a simplified selectivity index (SI) defined as the ratio (%) of cytotoxicity to cancer cells and normal cells at a given time point and particle concentration. This tendency can be confirmed by a simplified selectivity index (SI) defined as the ratio (%) of cytotoxicity to cancer cells to cytotoxicity to normal cells at a given time point and particle concentration. For MCNPs with [χTMA: χMUA]=80:20, the SI (48 h, 200 nM) was 19.55 for the HT1080/MEF pair and 8.86 for the MDA-MB-231/MCF-10A pair, whereas for purely cationic nanoparticles, the SI (48 h, 200 nM) was only 1.36 for the HT1080/MEF pair and 1.58 for the MDA-MB-231/MCF-10A pair, indicating that purely cationic nanoparticles show non-selective cytotoxicity.

Importantly, the selective cytotoxicity of 80:20 MCNP to cancer cells can be generalized to other types of cancer cells. A total of 13 cancer cell lines (including sarcoma, melanoma, breast cancer, prostate cancer and lung cancer) and 4 normal cell lines (including human fibroblasts) were tested in the same manner. The histogram in FIG. 1 is a summary of data. The selective death of cancer cells and limited cytotoxicity to normal cells is not simply because MUA thiol dilutes TMA, resulting in a decrease in net positive charge (dilution of charge).

To verify this, NPs were synthesized by replacing the MUA thiol with a straight-chain alkane C9 thiol, thus producing charge-diluted nanoparticles. It was confirmed that nanoparticles with [χTMA:χC9]=80:20 killed cancer cells, but the SI value was very close, indicating that the nanoparticles exhibit non-selective cytotoxicity (FIGS. 6c and 6d). Considering that nanoparticles with a smaller size (d=about 3 nm) did not show cytotoxicity even to cancer cells (FIGS. 8 and 9), it can be seen that the cancer cell-specific cytotoxicity of the MCNPs of the present invention is not simply due to charge dilution or the size of the nanoparticles.

### Example 3: Effect of MCNP Aggregation on Lysosomes

As previously described, the selectivity and aggregation behavior of mixed-charge nanoparticles are sensitively affected by the balance of the surface charge of the nanoparticles, and the use of fluorescent labels, a general approach to tracking the movement of nanoparticles in cells may affect the balance of the surface charge. Thus, in order to examine the movement of the nanoparticles produced in Example 1, the aggregation behavior of the MCNPs in living cells was monitored by label-free dark-field microscopy (DFM) (FIG. 10) and confocal-reflection microscopy (FIGS. 11 and 12). In addition, the microstructure of MCNP aggregates in the intracellular compartment was examined using TEM.

The total amount of MCNPs internalized into the cells was found to be similar between the cancer cells and the normal cells (FIGS. 10a and 13). However, DFM analysis showed a distinct difference in the aggregation behavior of the nanoparticles between the cancer cells and the normal cells over time (FIGS. 10b to 10e and 14). Inside the cancer cells, the MCNPs rapidly formed small clusters and merged into larger clusters to form very large aggregates with a diameter of 2 μm or more (FIGS. 10c and 10d, magenta curves). In contrast, in both normal cell lines (MEF and MCF-10A), the nanoparticles existed in the form of small clusters, and did not form large aggregates even after a long period of time.

In Example 1, based on the confirmation that pH-dependent assembly of MCNPs into an aggregate having a size suitable for endocytosis and macropinocytosis is possible, it was expected that, even within a cell, the MCNPs would exhibit a similar aggregation behavior depending on the pH concentration gradient of the endo-lysosomal system. To confirm this expectation, Rab5a-emGFP was used to label the endosome, and the lysosome was labeled with the LAMP1-TagRFP fusion protein. Initially, MVBs (multivesicular endosomes, FIGS. 10f and 12b), which are Rab5a-positive (Rab5+) vesicles with a size of about 0.5 μm, accumulate MCNP aggregates with a size of about 320 nm, are centrally located, and deliver the aggregates to an almost immobilized expanded Rab5+/LAMP1+ endosome “hybrid” compartment. In cancer cells, the MCNPs finally move to expanded LAMP1+/Rab5-lysosomes to form large aggregates, and in particular, form crystalline nanoparticle aggregates in HT1080 cells (FIGS. 10f and 15). In contrast, in normal epithelial cells, small MCNP aggregates accumulate rapidly in dynamic LAMP1+ vesicles of about 0.5 μm, or accumulate relatively little in Rab5+ vesicles.

### Example 4: “Swelling” of Lysosomes

Next, the co-localization of nanoparticle aggregates with acidified lysosomal organs marked with LysoTracker Red was examined. In FIGS. 11a, 11b and 16 to 18, co-localization corresponds to a yellow region (merged red LysoTracker and green reflection/NP image), and FIG. 11c shows the results of quantifying the co-localization using Pearson's correlation coefficient.

The largest aggregates were observed in the lysosomes of cancer cells treated with MCNPs with [χTMA: χMUA]=80:20 or 91:9. In addition, it was confirmed that, due to the uptake of MCNPs, the mean diameter (DL) of cancer lysosomes increased by at least 50%, and even more significantly increased in MCF7 and SKBR3 cells. In contrast, in normal cells treated with MCNPs with [χTMA: χMUA]=80:20, only marginal increases in the mean diameter (DL) of cancer lysosomes were observed (FIGS. 11d and 19).

### Example 5: Selective Destabilization of Lysosomal Membranes

Because lysosome ‘swelling’ may be indicative of lysosomal membrane permeabilization (LMP), the present inventors assessed the impact of MCNPs on the integrity of lysosomal membranes by determining the leakage of lysosomal acridine orange (AO) into the cytoplasm upon photo-oxidation (Bio Protoc. 4, e1162 (2014); Autophagy 11, 1408-1424 (2015)).

The uptake of various types of produced MCNPs further sensitized lysosomes of both HT1080 and MDA-MB-231 cells to damage by photo-oxidation (FIGS. 11e and 11f; (indicated by the rise in green fluorescence in left). In particular, χTMA:χMUA=91:9 and 80:20 MCNPs were more effective at destabilizing cancer lysosomes than pure-TMA (100:0) or 61:39 nanoparticles. Normal cell lysosomes were not destabilized by the above-described MCNPs, which means that the MCNPs of the present invention can selectively induce destabilization of the lysosomal membranes in cancer cells (FIGS. 11e, 11f and 21).

To test if lysosomal MCNP aggregation can induce LMP, the present inventors performed a galectin puncta assay (J. Virol. 86, 10821-10828 (2012)) using the reporter cell lines MCF7-mAG-gal39 (breast carcinoma) and U2OS-mCherrygal341 (osteosarcoma). In contrast to siramesine (which induces robust LMP and concurrent appearance of numerous galectin puncta), there were few galectin3 spots in cells treated with MCNPs with χTMA:χMUA=80:20 under standard conditions. Only when cells containing MCNPs with χTMA:χMUA=80:20 were briefly exposed to additional laser light (638 nm), did galectin3 spots appear around nanoparticle clusters. Despite the absence of galectin spots, lysosomal membrane disruption was detected in about 48% of cancer lysosomes (HT1080) in TEM images (versus only 3.7% in MEFs) at about 24 hours of exposure to MCNPs with χTMA:χMUA=80:20 (FIG. 12g).

In addition, the present inventors further tested the role of lysosomes in cancer-specific toxicity by co-treating cancer cells with nanoparticles and inhibitors of lysosomal acidification (bafilomycin A (Baf) or chloroquine (Cq)) or with an autophagy inhibitor (3-methyladenine (3MA) (FIGS. 11g and 11h). For comparison, the present inventors also tested sensitivity to inhibitors that block intrinsic apoptosis (cyclosporine A (CA) and caspase 3/7 inhibitor Ac-DEVD-CHO). It was confirmed that both Baf and Cq (but not the other inhibitors) protected cancer cells from death induced by the MCNPs with χTMA:χMUA=80:20 (FIGS. 11g, 11h and 23). On the other hand, neither of the two lysosomal inhibitors (including Baf and Cq) protected cancer cells from death induced by pure-TMA NPs (FIGS. 11g and 11h).

### Example 6: Impact on Lysosome Movement

To assess whether MCNP aggregation within lysosomes affected their ability to survey the cytoplasmic space, the present inventors monitored lysosome movement by time-lapse microscopy (FIGS. 24 and 25). In the absence of nanoparticles, lysosome trajectories did not visually differ between cancer versus normal cells, featuring heterogeneous path structures with random/‘jiggly’ trajectories positioned juxtanuclearly and more directional trajectories in the cell periphery. However, the uptake and aggregation of MCNPs with χTMA χMUA=80:20 severely impeded the motion of cancer lysosomes, at the same time having no discernible effect on the motion of normal cells' lysosomes.

### Example 7: Nanoparticle Accumulation Versus Clearance

To determine in which lysosomal organelles (low-pH lysosomes (Chem. Mater. 28, 2348-2355 (2016)) or higher-pH autolysosomes (Annu. Rev. Physiol. 77, 57-80 (2015)) nanoparticles accumulated, the present inventors tracked autophagic organelles by using an LC3B-eGFP-tagRFP autophagy sensor. In the normal cells, MCNPs with χTMA:χMUA=80:20 were mostly found in autolysosomes, whereas in cancer cells these MCNPs were localized to both autophagosomes and autolysosomes (FIGS. 12c, 12e, 26 and 27). TEM images showing NPs in vesicles containing concentric membrane rings, so-called multilamellar bodies (MLBs), confirmed localization to autolysosomes in non-cancerous cells (FIG. 12b). The overall numbers of autolysosomes increased, while those of autophagosomes decreased with time, indicating activation of the autophagy process in normal (non-cancerous) cells, but, in cancer cells, the numbers of both autophagosomes and autolysosomes remained roughly constant, with a higher number of autophagosomes than autolysosomes, indicating inhibited autophagic flux (FIGS. 12d and 12f).

Finally, in normal cells (MEFs), about 50% of internalized 80:20 MCNPs were secreted into cell media (FIGS. 12h and 12i), indicating rapid clearance of these MCNPs from healthy cells (FIG. 8). On the other hand, exocytosis of the same nanoparticles by cancer cells (HT1080) was significantly less efficient as only about 20% of the internalized MCNPs were secreted within a similar time interval (about 6 hours).

### Example 8: Comprehensive Analysis of the Origin of Cancer Selectivity of MCNPs

Considering the properties and cancer cell selectivity of the mixed-charge nanoparticles of the present invention, as consistent with previous reports, unlike purely cationic TMA nanoparticles (χTMA:χMUA)=100:0), which exhibit non-selective cytotoxicity, mixed-charge nanoparticles (MCNPs) with a positive net charge exhibit cancer cell-specific cytotoxicity that is mediated gradually from the cell surface to the inside of the cell. Due to their pH-dependent aggregation, MCNPs accumulate in cancer lysosomes, destabilize the lysosomes, and impair the function of cancer lysosomes, resulting in cancer cell death.

It is understood that the difference in cancer selectivity between MCNPs and pure TMA nanoparticles (100:0) is due to the difference in pH-specific aggregation ability. Ex vivo aggregation experiments on nanoparticles confirmed in Example 1 indicated that MCNPs, including MCNPs with (χTMA:χMUA) of 80:20, form numerous intermediate-sized (about 50 to 100 nm) clusters at a pH of about 5.5 to 7 (FIG. 3d), whereas purely cationic nanoparticles sharply transition to very large aggregates abruptly at about pH 6, and any intermediate-size aggregates are much less numerous. This aggregation behavior is consistent with the aggregation behavior of MCNPs that form clusters on the surfaces of cancer cells (extracellular pH=6.5, FIG. 10f).

Intermediate-sized clusters from MCNPs exhibit a size very suitable for endocytosis, and are mostly internalized into cells by endocytosis. While transiting the increasingly more acidic compartments of the endo-lysosomal tract, the small clusters initially formed at the cell surface coalesce into larger aggregates and eventually form crystals inside lysosomes (FIGS. 10f and 15).

On the other hand, the purely cationic NPs, in addition to endocytosis, are internalized via direct membrane penetration, which is known to cause membrane permeabilization and indiscriminate cytotoxicity (FIG. 26 and Table 1).

TABLE 1

| χ_{TMA}:χ_{MUA} | | 80:20 | 100:0 |
|---|---|---|---|
| | Evidence | [+/−] NPs | [+] NPs |
| Selective killing of cancer cells | *Cytotoxicity | √ | − |
| Lysosomal cell death | *Cytotoxicity with | √ | − |
| | inhibitors | | |
| Uptake through endocytosis | *Live cell imaging | √ | √ |
| Massive aggregation | *TEM | √ | − |
| in lysosomes | *Live cell imaging | | |
| Lysosomal swelling | *Live cell imaging | √ | − |
| Inhibition of lysosome movements | *Live cell imaging | √ | − |
| Uptake through membrane | *TEM | − | √ |
| penetration | | | |
| Cytoplasmic aggregation | *TEM | − | √ |
| | *Live cell imaging | | |

The initial stages of the aggregation process can be facilitated by the protein corona on the negatively charged nanoparticles in the presence of serum proteins (FIGS. 4, 29 and 30). By lowering the pH, proteins' acidic residues in the corona can be protonated to reduce charge-charge repulsions and facilitate aggregation via hydrogen bonding and/or van der Waals forces. This scenario, however, does not explain the eventual formation in the lysosomes of the tightly packed (and in HT1080 cells surprisingly crystalline) NP assemblies in which the gaps between neighboring particles are only about 2.4 nm, which is roughly twice the thickness of the nanoparticle monolayers (FIGS. 10f and 15).

On the other hand, nanoparticles in such aggregates/crystals are experiencing electrostatic repulsions between TMA ligand head groups. These repulsions can be offset by several factors, including relatively strong (about 10 kT) van der Waals interactions between proximal Au cores, the formation of hydrogen bonds between protonated MUA groups or screening due to relatively concentrated ions present in the lysosomes (about 0.5 mM Ca²⁺ and 80 mM Cl⁻). In addition, confinement, a gradual increase in the volume fraction, and entropic effects might be at play. In particular, the gain in conformational entropy of protein fragments cleaved off the protein coronas by proteases can drive aggregation of nanoparticles, akin to small colloids causing aggregation of larger ones present in the mixture (FIGS. 3e and 5).

On the other hand, as shown in FIG. 2c, it was shown that ex vivo cathepsin D enhanced MCNP aggregation, and in cells, serine proteases would play this role (FIG. 31).

### Example 9: Impact of Protein Corona in MCNP Internalization Stage

Protein corona is a protein shell adsorbed on nanoparticles, and can be classified into a hard corona and a soft corona, which are composed of high-affinity and low-affinity proteins, respectively. Characteristics of the corona, such as final composition and formation time, depend on various factors such as the size, shape, material, and surface properties of nanoparticles, as well as the properties of the solution (pH, ionic strength, temperature), the type and concentration of proteins present (e.g., protein coronas formed in 10% serum have less protein with a pI>8 than those formed in 50% serum solutions).

Although the presence of the protein corona can reduce the direct interaction between the nanoparticles of the present invention and the cell membrane, this reduction in intracellular internalization efficiency has been reported to occur only in NPs larger than 50 nm, and it was reported that there was no effect on the caveolin-mediated intracellular uptake level of nanoparticles of about 5 nm (ACS Nano 10, 4421-4430, 2016). Strategies to avoid non-specific binding of proteins include pre-adsorption of polyethylene glycol (PEG, higher MW, less protein adsorption) and other nonionic polymers (ACS Nano 6, 9182-9190, 2012), zwitterions (Langmuir 23, 12799-12801, 2007), mixed-charge self-assembled monolayers (SAMs) (Langmuir 27, 5242-5251, 2011 (trimethylammonium+sulfonic acid)) or albumin (Int. J. Nanomedicine 12, 3137-3151, 2017).

To compare the protein corona between nanoparticles with χTMA:χMUA ratios of 100:0, 80:20 and 0:100, respectively, SDS-PAGE was used to examine the protein in the corona, and BCA (bicinchoninic acid) assay was used to quantify the total amount of protein. As a result, it was confirmed that MCNPs with χTMA:χMUA of 80:20 adsorbed less protein than pure TMA nanoparticles with χTMA:χMUA of 100:0 (FIG. 29c). In addition, it was confirmed that the protein corona in MCNPs with χTMA:χMUA of 80:20 was composed of less diverse proteins than pure TMA with χTMA:χMUA of 100:0 or pure MUA with χTMA:χMUA of 0:100 (FIG. 29). The most abundant protein in the MCNP with χTMA:χMUA of 80:20 was a protein with a molecular weight of about 60 kDa, and the pure TMA nanoparticles showed various protein bands with low and high molecular weights. Considering the type and molecular weight of the existing serum protein, it is expected that the proteins adsorbed to the MCNPs of the present invention will be about 67 kDa bovine serum albumin (BSA) and/or about 63.5 kDa fibrinogen a chain, IgG (about 25 kDa+50 kDa) or apolipoproteins such as ApoA1 (28 kDa).

In addition, uptake in lysosomes in HT1080 and MEF cells (FIGS. 30a and 30e, respectively) and accumulation of nanoparticles were similar in the presence or absence of serum proteins in culture media (FIGS. 30b and 30f). On the other hand, cytotoxicity increased in the serum-free state (FIGS. 30d and 30h), which is expected to be due to a lack of nutrients.

Summarizing the results of this example, it can be seen that the formation of a protein corona in MCNPs due to the presence of serum proteins does not significantly affect cellular internalization and lysosomal accumulation and aggregation.

### Example 10: Biochemical Effect of MCNPs and Comparison with Existing LMP Inducing Drugs

The inner lysosomal membrane contains a high concentration of anionic phospholipid bis(monoacyl phosphoglycerol)phosphate (BMP), which functions as a docking lipid for various enzymes including lysosomal sphingomyelinase. Sphingomyelinase supports the integrity of the lysosomal membrane by converting sphingomyelin into ceramide. On the other hand, acidic ceramidase converts sphingolipid ceramide into sphingosine, which can leave the lysosome, unlike ceramide. Cationic amphiphilic drugs (CADs), for example, siramesine, replace acidic sphingomyelinase in anionic BMP, and induce potent LMP by promoting proteolysis, accumulation of sphingomyelin, and destabilization of the lysosomal membrane (Nat. Rev. Drug Discov. 1, 491-492 (2002)). On the other hand, the MCNPs of the present invention exhibit LMP induction through aggregation in lysosomes, unlike conventional cationic amphiphilic drugs. The MCNP aggregation effect of the present invention occurs more gradually (FIG. 11) and is not related to acidic sphingomyelinase (FIG. 32).

On the other hand, the accumulation of lysosomal ceramides that self-associate into microdomains that disrupt the lamellar tissue of the membrane bilayer at high concentrations affects the integrity of the membrane and makes the membrane less mechanically robust (Sci. Adv. 4, eaau3546 (2018)). From this point of view, the level of acidic ceramidase in normal cells increases in response to the MCNPs of the present invention, which prevents the accumulation of ceramides and can play a protective role (FIG. 32). In addition, the lysosomal protein showed a distinct difference in response to the crystallization of MCNPs in cancer cells and normal cells (FIG. 33). Specifically, mTORC1 (mammalian target of rapamycin complex 1), a signaling complex involved in cell growth, autophagy, and metabolic regulation, was cleared from the surface of cancer lysosomes containing nanoparticles. In contrast, it was confirmed that the association between lysosomes and mTORC1 rather increased in normal cells (FIG. 34).

### Example 11: Change in Osmotic Pressure by MCNPs in Cancer Cells

In fact, three methods were used to determine the volume fraction (η) of nanoparticles.

First, the lower limit of the volume fraction in the measurement of uptake was calculated (blue arrow on the left of FIG. 35).

Second, approximate volume fractions were estimated using TEM images. The present inventors determined the region without nanoparticles, the NP region packed with about 3-layer thickness, and the darkest region presumed to have the same thickness as the microtome slice (80 nm, FIG. 35a). Since microtome slices are randomly positioned with respect to lysosomes, the NP volume fraction of the slice represents a representative value of the lysosomal fraction. The values obtained in this way are displayed as a bar graph at the bottom of FIG. 36 and used for estimation of the number of NPs per lysosome.

Third, when all the NPs added to cell incubation are internalized into lysosomes, the NP volume ratio of the lysosomes is a value between 0.17 and 0.58. In FIG. 36, this value is indicated by orange horizontal brackets. It was assumed that the volume fraction of NPs could not exceed the volume fraction of dense spheres (0.74).

Estimation of the number of nanoparticles per single lysosome was performed as follows. After estimating the volume fraction of NPs in the microtome slice, the volume of the corresponding lysosome was estimated. The lysosome was assumed to be spherical, and it was considered that the radius (r_{c}) of any cross-section of a sphere (microtome slice) was always smaller than the central radius (R) of the sphere (FIG. 35b). More precisely, the average value <r_{c}> is equal to R·π/4. This is because the distance D from any cross-sectional plane to the center of the sphere is uniformly distributed between 0 and R. On the other hand, r_{c} at a given d is as in Equation 1 below.

r_{c}(D)=\sqrt{R²−D²} [Equation 1]

Therefore, the integral value for D from 0 to R of the r_{c}(D) is equal to the ¼ area of the circle that is the lower area of the graph in FIG. 35C, and thus the average <r_{c}> is as shown in Equation 2 below.

$$the average 〈 {r}_{2} 〉 = \frac{1}{n} {\int }_{0}^{R} {r}_{c} ( D ) dD = \frac{1}{R} {\int }_{0}^{R} \sqrt{{R}^{2} - {D}^{2}} dD = \frac{1}{R} , π R / 4 = π / 4 \qquad [ Equation 2 ]$$

The geometric correction indicates that the effective apparent radius (r_{c}=√A/π) of the lysosomal microtome section calculated in the apparent area A of the lysosome in the TEM image differs by π/4 from the effective radius calculated using confocal fluorescence microscopy. This difference is due to the fact that the microtome slice (80 nm) is much thinner than a typical lysosomal diameter (approximately 1 μm, FIG. 35d), although the axial size of the confocal collection volume is similar to the typical lysosomal diameter. Therefore, the apparent radius of the lysosome in the TEM image is 4r_{c}/π, an unbiased estimate of the lysosomal radius with the r_{c} plane. Therefore, the volume (V) thereof is as shown in Equation 3 below.

V=4π(4r_{c}/π)³/3 [Equation 3]

The calculated volume fraction (η) of NPs within the imaged microtome slice represents the NP volume fraction in the total lysosome volume (FIG. 35a). Therefore, the total number of NPs in a given lysosome can be calculated as ηV/V_{NP}. Here, V_{NP}=4π(r_{NP})³/3. FIG. 35f shows the numbers of NPs internalized into HT1080, MDA-MB-231 and MEF in comparison with the number of NPs in microtome sections (FIG. 35e), when incubated with 50 nM MCNPS (χTMA: χMUA=80:20) for 24 hours.

Next, the present inventors assessed how the osmotic pressure generated at the boundary of the lysosomal membrane by the aggregation of MCNPs inside the cancer lysosome can contribute to the destruction of the lysosome. Liu et al. reported that lysosomes can be disrupted under an osmotic pressure between 1.41 atm and 22 atm estimated by the van't Hoff formula using lysosomal diameters of 2 μm and 0.8 μm, respectively, and this disruption can be inhibited by incubating the cells in an additional 100 mM NaCl solution corresponding to an additional 5 atm osmotic pressure (J. Mater. Chem. B 2, 3480-3489 (2014)). Based on this report, disruption of osmotic pressure begins when an osmotic pressure gradient between about 1.4 atm and 5 atm occurs. The amount of mixed-charge NPs that must be present in the lysosome to induce a difference in osmotic pressure corresponding to the corresponding range can be estimated as follows.

When nanoparticles have a total charge of e*Z, some of the counterions of Z are free-floating, while others condense more on the surface. Therefore, in the concentration (C_{NP}) and volume of the charged nanoparticles, the osmotic pressure at temperature T is between the C_{NP}K_{B}T value, which is the osmotic pressure of one nanoparticle, and the pressure value (1+Z) C_{NP}K_{B}T (where K_{B} is the Boltzmann constant) due to all counterions. However, in this case, an error may occur. Since accurate calculation of osmotic pressure (Eur. Phys. J. B 1, 337-343 (1998); Trans. Faraday Soc. 66, 500-508 (1970); J. Chem. Phys. 23, 1057-1068 (1955)) is obtained by evaluating the concentration of ions between NPs at the edge of Wigner-Seitz cells, the Poisson-Boltzmann (PB) equation must be solved (30). However, since the exact electrolyte concentration of the lysosome was not known, a solution having an electrolyte concentration of about 10% or less was used instead in the experiment (31; 32). The osmotic pressure gradient bounded by the lysosomal membrane was obtained by the following Equation 4.

$$[ Equation 4 ]$$

$$Δ \prod = {k}_{B} T ( {c}_{+} ( {R}_{w} ) + {c}_{-} ( {R}_{w} ) - 2 {c}_{s} ) = 4 {k}_{B} {Tc}_{s} {sh}^{2} ( \frac{ψ ( {R}_{w} )}{2} )$$

wherein C₊ (r) and C₋ (r) are the concentrations of cations and anions at diameter r from the particle center, Cₛ is the salt concentration of the medium, and Rw is the radius of the Wigner-Seitz cells. When the diameter of the nanoparticle is “a” and the volume fraction of the nanoparticle is η, RW=aη^{-1/3}.

The reduction in electrostatic potential was obtained using Equation 5 below.

$$ψ ( r ) = \frac{e Ψ ( r )}{{k}_{B} T} \qquad [ Equation 5 ]$$

wherein ψ(r) is the electrostatic potential, and r is the distance from the center of the nanoparticle. The potential is a solution of the nonlinear PB equation and must be calculated numerically. However, the linearization of the above equation is derived from a simple analytical solution of the Yukawa form as shown in Equation 6 below.

$$ψ ( r ) = {Z}_{eff} {l}_{B} \frac{exp [ κ ( a - r ) ]}{r ( 1 + κ α )} \qquad [ Equation 6 ]$$

wherein Z_{eff} is the effective charge, and l_{B}=e2/(4πE∈T) is the Bjerrum length. Here, ∈ is the dielectric constant of water, and k=√(8πL_{B}N_{A}I₀) is the Debye screening length and depends on the ionic strength I₀. Thus, it is affected by Cs. A charge renormalization approach was used to replace the effective charge with a saturation value (4Ka(1+κa)/1b), where K is about 1, somewhat dependent on η. A screening coefficient κ* was introduced into the solution of Equation 7 below to accurately include the dependence of K on η.

$$[ - 1 + {f}_{+} \frac{\text{?}}{a} + {f}_{-} \frac{\text{?}}{a} ] \sqrt{1 - {( \frac{κ}{\text{?}} )}^{4}} = 4$$

$$\text{?} \text{indicates text missing or illegible when filed}$$

wherein f_{±}=[(κ_{*}R_{W}±1)/2κ_{*}]exp(±κ_{*}R_{W}) and depends on η by RW. The above equation was solved numerically for κ* using a simple Newton procedure. Equation 4, which is an osmotic pressure equation, can be expressed as a screening coefficient κ* as shown in Equation 8 below.

$$Δ \prod = \frac{{k}_{B} T ( {κ}_{*}^{2} - {κ}^{2} )}{4 π {l}_{B}} \qquad \{ Equation 8 ]$$

The osmotic pressure calculated using the above approach ignores van der Waals forces, meaning that the NPs are not in a highly aggregated state. Although this assumption can be understood as contradicting the appearance of dense NPs in TEM images (FIGS. 15, 35a and 10f), these TEM images show the final state within the lysosome, where the osmotic pressure in the process of NP uptake by the lysosome before beginning of aggregation is estimated. Indeed, early-stage TEM images of NPs in multivesicular endosomes/multivesicular bodies (MVBs) (FIGS. 10f and 12) indicate that aggregation of NPs is a gradual process. The compact aggregation seen in late-stage TEM images (FIG. 35a) may have been increased in the labeling and cell fixation processes performed prior to TEM imaging, but confocal testing showed that these results were not artificial and may occur even in living cells in the same manner.

The blue curve in FIG. 36 shows the dependence of the osmotic pressure Δπ on the volume fraction q calculated using Equation 8 (a=(2*1.5 nm+5.2 nm)/2, T=309K), and the ambient concentration of ion is the same as that in the buffer solution (PBS, 280 to 320 mOsm/L). On the other hand, in the case of normal cells, rapid disruption of lysosomes started at an osmotic pressure of about 1.4 atm. This limit is indicated by horizontal dashed lines in FIG. 36. Slow uptake of NPs allows the lysosomes to have sufficient time to react, and gradual swelling rather than rapid disruption may begin long before the pressure differential reaches 1.4 atm. On the other hand, the pressure across the membrane from the tension of the stable membrane is estimated to be at least 3×10⁻³ atm, which is indicated by the dotted line in FIG. 36.

It was confirmed that the increase in osmotic pressure of lysosomes due to NP uptake did not reach about 1.4 atm, a pressure capable of destroying lysosomes in healthy cells even after 24 hours. On the other hand, lysosomes in malignant cells have a weaker membrane than normal cells, and thus can be disrupted at the critical pressure between the dashed lines shown in FIG. 36. In particular, it can be seen that the pressure after 24 hours after MCNP uptake reaches this critical pressure. As shown in the box plots at the bottom of FIG. 36, the measured volume fractions are widely distributed, and thus it can be inferred that, even if the pressure calculated for the median volume fraction (orange circle) does not exceed the critical level, at least some lysosomes in the population will be destroyed.

Since the charge renormalization approach essentially ignores the effect of the actual charge of nanoparticles on the osmotic pressure, the increase in osmotic pressure due to the accumulation of MCNPs in cancer lysosomes is expected to be higher than the value expected by the present inventors.

### Example 12: Physical Effect of MCNPs

MCNPs exhibit ionicity and are surrounded by counter ions (Nat. Nanotechnol. 11, 603-608 (2016)), and thus when MCNPs enter lysosomes, they may show results similar to those of the addition of a large amount of inorganic salt, causing osmotic pressure differences, resulting in osmotic flow and lysosomal swelling. The amounts of nanoparticles accumulated in cancer lysosomes, calculated through the electrostatic model in the Example of the present invention, are as follows: up to 27% [v/v] for HT1080 cells, about 45% vs. 75% full packing for MDA-231; FIGS. 35 and 36).

Considering the amount of these nanoparticles, it can be seen that the aggregation of MCNPs in cancer cell lysosomes and the resulting osmotic pressure difference are sufficient to induce osmotic swelling of the lysosomes, but do not directly disrupt the lysosomes. This is also consistent with the results of the Examples. In normal cells, this osmotic effect by MCNPs is significantly reduced due to clearance into autolysosomes, limited aggregation, and MCNP release due to exocytosis.

Materials and Methods

Nanoparticle Synthesis and Chemicals

Auric acid (HAuCl4·3H2O) was purchased from AlfaAesar (cat no. 36400) and dilauryl dimethylammonium bromide (DDAB) from TCI America (cat no. 3282-73-3). The following reagents were purchased from Sigma-Aldrich: dodecyl amine (DDA, cat no. D222208), hydrazine monohydrate 64% (cat no. 207942), tetrabutylammonium borohydride (TBAB, cat no. 230170), tetramethylammonium hydroxide 25% (TMAOH, cat no. 331635), 1-nonanethiol (C9, cat no. 674273) and 11-mercaptoundecanoic acid (MUA, cat no. 450561). N,N,N-trimethyl(11-mercaptoundecyl)ammonium chloride (TMA; cat no. FT #006) was purchased from ProChimia Surfaces (Poland). Toluene (cat no. AH347-4), methanol (cat no. AH230-4) and acetone (cat no. AH010-4) were purchased from Honeywell. Dichloromethane (amylene-stabilized, cat no. 39116) was purchased from AlfaAesar. Cellulose acetate (CA) 0.2 μm sterile filters (cat no. 13CPO20AS) were purchased from ADVANTEC (Japan)

Cell Culture and Biologicals

Human fibrosarcoma HT1080 (cat no. CCL-121), human breast epithelial cells MCF-10A (cat no. CRL-10317) and human breast adenocarcinoma MDA-MB-231 (cat no. HTB-26) and MDA-MB-468 (cat no. HTB-32), Rat2 fibroblast cell line (cat no. CRL-1764), human skin fibroblasts CCD1058SK (cat no. CRL-2071), human breast adenocarcinoma MCF7 (cat no. HTB-22), human breast adenocarcinoma SK-BR-3 (cat no. HTB-30) and mouse melanoma B16F1 (cat no. CRL-6323) were obtained from the American Type Culture Collection (ATCC). Prostate carcinoma LNCaP.FCG (cat no. 21740), human malignant melanoma A375P (cat no. 80003), human breast ductal carcinoma BT474 (cat no. 60062) and HCC38 (cat no. 950038) and lung cancer cell lines A549 (cat no. 10185), NCI-H1299 (cat no. 91299) and NCI-H1573 (cat no. 91573) were obtained from the Korean Cell Line Bank (http://cellbank.snu.ac.kr/english/index.php). MEFs were a gift from X. Tong (Northwestern University Medical School, Chicago, Ill., USA). Galectin3 reporter cell lines MCF7-mAG-gal39 (breast carcinoma) and U2OS-mCherrygal3 (osteosarcoma) were generously provided by D. Egan and H. Wodrich, respectively. Cell lines have not been independently authenticated. Detailed information on each cell line is shown in Table 2 below.

TABLE 2

| | | | | | | | | | | |
|---|---|---|---|---|---|---|---|---|---|---|
| | | | | | | Tumor | TP53 | TP53 prot | Other | Cell culture |
| Cell line | Type | ER | PR | HER | Source | type | states | sequence | mutation | media |

NON-CANCEROUS CELLS

| | | | | | | |
|---|---|---|---|---|---|---|
| MCF-10A | [symbol] | [symbol] | [symbol] | WT | — | [symbol] |
| CCD [symbol] | [symbol] | [symbol] | N | WT | — | [symbol] |
| Rat2 | [symbol] | [symbol] | N | WT | — | [symbol] |
| MEF | [symbol] | [symbol] | N | WT | — | [symbol] |

BREAST CANCER

| | | | | | | | | | | |
|---|---|---|---|---|---|---|---|---|---|---|
| MCF7 | Lu | [symbol] | [symbol] | | [symbol] | AC | WT | — | [symbol] | [symbol] |
| SKBR3 | Lu | [symbol] | [symbol] | [symbol] | [symbol] | AC | [symbol] | [symbol] | [symbol] | [symbol] |
| [symbol] | Lu | [symbol] | [symbol] | [symbol] | [symbol] | [symbol] | [symbol] | [symbol] | [symbol] | [symbol] |
| HCC38 | [symbol] | [symbol] | [symbol] | [symbol] | [symbol] | [symbol] | [symbol] | [symbol] | | [symbol] |
| MDA-MB-168 | [symbol] | [symbol] | [symbol] | [symbol] | [symbol] | [symbol] | [symbol] | [symbol] | [symbol] | [symbol] |
| | | | | | | | | | [symbol] | |
| MDA-MB-231 | [symbol] | [symbol] | [symbol] | [symbol] | [symbol] | [symbol] | [symbol] | [symbol] | [symbol] | [symbol] |
| | | | | | | | | | [symbol] | |
| | | | | | | | | | [symbol] | |

LUNG CANCER

| | | | | | | |
|---|---|---|---|---|---|---|
| [symbol] | Lung | Ca | WT | — | [symbol] | [symbol] |
| | | | | | [symbol] | |
| [symbol] | [symbol] | [symbol] | [symbol] | [symbol] | [symbol] | [symbol] |
| | | [symbol] | | | | |
| [symbol] | [symbol] | [symbol] | [symbol] | [symbol] | [symbol] | [symbol] |
| | [symbol] | [symbol] | | | [symbol] | |

OTHER CANCERS

| | | | | | | |
|---|---|---|---|---|---|---|
| HT1080 | [symbol] | FS | WT | — | [symbol] | [symbol] |
| | | | | | [symbol] | |
| | | | | | [symbol] | |
| [symbol] | [symbol] | Ca | WT | — | [symbol] | [symbol] |
| | [symbol] | | | | [symbol] | |
| | | | | | [symbol] | |
| [symbol] | [symbol] | [symbol] | WT | — | [symbol] | [symbol] |
| | | | | | [symbol] | |
| [symbol] | [symbol] | [symbol] | WT | — | | [symbol] |

[symbol]

indicates data missing or illegible when filed

Cell Culture

MEF, HT1080, Rat2 and B16F1 cells were cultured in Dulbecco's modified Eagle medium (DMEM) supplemented with 10% FBS and 25 μg/ml gentamycin. MCF-10A cells were cultured in DMEM/F12 supplemented with 5% horse serum, 20 ng/ml EGF, 10 μg/ml insulin, 0.5 mg/ml hydrocortisone, 100 ng/ml cholera toxin and penicillin/streptomycin (10,000 units/ml penicillin and 10,000 μg/ml streptomycin). CCD1058SK and A375P were cultured in MEM supplemented with 10% FBS and 25 μg/ml gentamycin. MCF7, SK-BR-3, HCC38, NCI-H1299, and LNCaP.FCG cells were cultured in RPMI supplemented with 10% FBS and 25 μg/ml gentamycin. BT474 and NCI-H1573 cells were cultured in RPMI supplemented with 5% FBS and 25 μg/ml gentamycin. A549 cells were cultured in F-12K supplemented with 10% FBS and 25 μg/ml gentamycin. All cell lines, except MDA-MB-231 and MDA-MB-468, were cultured in a 5% CO₂ atmosphere at 37° C. MDA-MB-231 and MDA-MB-468 cells were cultured in L-15 medium supplemented with 10% FBS and 25 μg/ml gentamycin at 37° C. without CO₂ (in air atmosphere), as specified by ATCC. All cell lines were routinely tested and verified as mycoplasma-free.

Cytotoxicity Assay

The killing of cells by MCNPs was quantified using a LIVE/DEAD Viability/Cytotoxicity Kit for mammalian cells based on staining live cells with calcein-AM (0.5 μM) and dead cells with ethidium homodimer-1 (1 μM) dyes.

Data shown in FIG. 1 correspond to experiments in which medium was removed and replaced with fresh medium containing χTMA:χMUA=80:20 MCNPs (200 nM) (200 nM), and cells were incubated for 24 h for HT1080, MEF, Rat2 and CCD1058SK (sarcoma and normal fibroblast); for 47 h for MDA-MB-231, MCF-10A, MCF7, SK-BR-3, B16F1 and A375P (breast carcinoma, melanoma versus normal epithelial cells); for 72 h for HCC38, MDA-MB-468, LNCaP-FCG and BT474 (breast and prostate cancer); or for 96 h for A549, NCI-1299 and NCI-H1573 cells (lung cancer) at 37° C. in respective incubators.

To assess acute cytotoxicity in detail, medium was removed and replaced with fresh medium containing MCNPs in concentrations of 50, 100 or 200 nM. Then, cells were incubated for 24 h for HT1080 and MEF or for 48 h for MDA-MB-231 and MCF-10A at 37° C. in respective incubators (FIG. 6). To assess the long-term cytotoxicity, cells were treated for 48, 72 or 96 h with 50 nM MCNPs (FIG. 6, insets). Control represents cells cultured without nanoparticles. Because MCF-10A cells were not appreciably labeled with calcein-AM, they were instead pre-labelled with CellTracker Green CMFDA dye (10 μM) for 30 min at 37° C. before MCNP treatment, then washed once with PBS, treated with nanoparticles and finally labelled with ethidium homodimer-1 dye (1 μM). Images of live (green) and dead (red) cells were automatically acquired using an IncuCyte ZOOM system fitted with a ×20 objective lens (Live-Cell Analysis System IncuCyte® ZOOM, Essen BioScience, Inc.). The numbers of live/dead cells were counted using the Cell Counter PlugIn for ImageJ software (NIH, MA, USA) and the cytotoxicity was expressed as the percentage of dead cells.

Quantification of Cellular Uptake of Mixed-Charge Nanoparticles by ICP-AES

Au nanoparticle uptake in cells was determined with Inductively Coupled Plasma Atomic Emission Spectroscopy (ICP-AES, Varian 700-ES, USA). The amount of Au in samples was normalized against the number of cells per sample. Cells were counted using the Countess II FL Automated Cell Counter (ThermoFisher Scientific).

Live Cell Imaging

For all live cell imaging experiments, cells were seeded in native medium into glass-bottomed cell culture dishes coated with fibronectin (25 μg/ml) and allowed to adhere at 37° C. overnight. Cells were then cultured untreated (Control/t=0) or were treated with [+/−] NPs (50 nM, unless stated otherwise) and cultured for the indicated time intervals at 37° C. Live cell imaging was performed on a Nikon AR1 Confocal microscope fitted with a ×60, 1.4NA (numerical aperture) or ×100, 1.45NA oil immersion objective and LU-NV laser unit (Nikon) housing 488 nm (for exciting emerald green fluorescent protein, eGFP), 561 nm (RFP/LysoTracker Red DND-99) and 638 nm (for confocal reflection mode) laser lines. In most experiments, the confocal reflection mode was combined with simultaneous fluorescence imaging of small-molecule dyes/fusion proteins, thus allowing us to track and co-localize MCNP aggregates with various cellular structures/markers in living cells with high spatiotemporal resolution. Endosomes were labelled with Rab5a-eGFP fusion protein and lysosomes with LAMP1-TagRFP fusion protein (which marks lysosomes independently of their acidification status, but, similar to Lysotracker, may also mark autolysosomes) 24 h before treatment with the nanoparticles. In separate experiments, APs and ALs were tracked using an LC3B-eGFP-tagRFP autophagy sensor. Here, APs correspond to neutral vesicles marked with LC3B protein (in neutral pH, both eGFP and tagRFP emit light, so eGFP+/tagRFP+ double-positive vesicles are classified as APs) and ALs to acidified vesicles marked with LC3B protein (in an acidic environment, pH-sensitive eGFP is quenched, so eGFP−/tagRFP+ vesicles are classified as ALs). Time-lapse images were acquired at about 200 ms intervals (about 5 frames per second). For co-localization and lysosomal diameter analysis, lysosomes (including lysosomes, autolysosomes and lysosomal vacuoles) were labelled with LysoTracker Red DND99 (50 nM) for the last 30 min of NP treatment and high-resolution midplane x-y images of single cells were acquired immediately using the confocal microscope galvano scanner mode. Temperature and CO2 concentrations were maintained using a stage-fitted incubator system and gas mixer from Live Cell Instruments. Unless otherwise specified, all cells were imaged in their native medium and respective CO2 levels.

Determination of Lysosomal Membrane Integrity

Loss of lysosomal membrane integrity was determined by live cell imaging of the leakage of lysosomal AO into the cytoplasm in response to photo-oxidation, as described previously by Petersen et al. Briefly, cells cultured in glass-bottomed cell culture dishes were treated with MCNPs (50 nM) for 6 h (HT1080 and MEF) or 12 h (MDA-MB-231 and MCF-10A). Following treatment with NPs, AO (2 μg/ml) was added directly to the growth medium for an additional 15 min at 37° C. Cells were washed three times, medium was replaced with PBS containing 3% FBS, and samples were transferred to a Nikon AR1 laser scanning confocal microscope stage equipped with a ×60, 1.4NA oil immersion objective. AO was excited using 488 nm light from a laser diode (at 10% laser intensity) and lysosomal membrane disruption and leakage of AO into cytoplasm were monitored by acquiring time-lapse images every 500 ms for a total period of 120 s in a channel defined by a 495-555 nm bandpass filter. Green fluorescence intensity was quantified within regions of interest (ROIs), marking groups of cells by using NIS-Elements software. The galectin puncta assay was performed as described IN Virol. 86, 10821-10828 (2012).

Transmission Electron Microscopy

Cells were exposed to 50 nM [+/−] NPs (χTMA:χMUA=80:20) for 1 h, 6 h or 24 h and then washed with PBS, fixed with 2% glutaraldehyde and 2% paraformaldehyde solution in PBS-sodium cacodylate buffer (50:50). Cells were harvested and post-fixed with 2% osmium tetroxide, stained with 2% uranyl acetate and embedded in propylene oxide and Eponate. Sections, 80 nm thick, were cut with a Leica ultracut UCT ultramicrotome, and samples were examined on Zeiss LEO 912AB or JEM2100 (JEOL, Korea) electron microscopes at the Korea Basic Science Institute (KBSI, Chuncheon Center, Chuncheon City, South Korea).

Data Analysis

Unless stated otherwise, all image processing and analysis was performed using NIS-Elements Imaging Software v. 4.50 (Nikon, Japan).

Quantification of Co-Localization

Co-localization corresponds to yellow regions (merging of red LysoTracker Red and green reflection/NP images) and was quantified by computing the PCC37 using NIS-Elements software. First, the background was subtracted for the two images (reflection=nanoparticle aggregates and Lysotracker Red=lysosomes) and ROIs were drawn around single cells (the cell outline was identified in the differential interference contrast (DIC) image). For co-localization of nanoparticle aggregates with autophagic organelles, PCC values were determined for Reflection/eGFP (signifying co-localization with APs) and Reflection/TagRFP (signifying co-localization with APs and ALs) images, as described above. Based on PCC (PCC>0.1 was treated as positive co-localization), each cell was classified as having NP aggregates localizing only in yellow (AP), only in red (AL), in both types of vesicle (AP+AL) or in neither type of vesicle. Proportions of cells displaying the indicated co-localization pattern are shown in FIG. 12.

Quantification of Lysosome Size

Lysosomal size was analyzed by quantifying the mean diameter of LysoTracker Red DND-99-labelled vesicles from X-Y midplane images obtained with confocal microscopy. Lysosomes were identified by searching for threshold numbers until threshold masks became the same size as the lysosomes. Because the vesicles were not perfectly spherical, the equivalent diameter (D_{eq}) defined as the diameter of a circle with the same total area as the measured object was calculated:

Deq=√(4×area/π).

Vesicles of diameters ranging from 0.3 μm to 3 μm were used for analysis and clustered lysosomes were excluded in the size analysis. Lysosomal diameter is expressed as percent increase in diameter relative to control (untreated/t=0).

Statistics and Reproducibility

Data were analyzed using Microsoft Excel and OriginPro software. Data in bar graphs and line plots are presented as mean±SD, unless specified otherwise. For data displayed in box-and-whisker plots, boxes delineate lower and upper quartiles of the data, middle lines show median values, dots (if present) show individual data points, and whiskers show minimal and maximal values for each dataset.

A two-tailed Student's test was used for comparisons of two groups and one-way ANOVA followed by Tukey's post-hoc test for multiple comparisons. Exact P values, as appropriate, are shown in the figures, listed in the figure legends or, for FIG. 6, listed below when P≥0.00001. Significant differences are denoted with asterisk(s) as follows: *P<0.05, **p<0.00001. Results were considered significantly different when P p<0.05. *p<0.05, **p<0.00001.

### INDUSTRIAL APPLICABILITY

The mixed-charge nanoparticles of the present invention are specifically localized and crystallized in cancer cell lysosomes through pH-dependent aggregation behavior due to the balance of positively charged ligands and negatively charged ligands decorated on the surface of the nanocore, and may induce lysosomal membrane permeabilization (LMP) and LMP-mediated lysosomal cell death, like cationic amphiphilic drugs (CADs). Since the nanoparticles of the present invention exhibit a cancer cell-specific killing effect, they may overcome the limited medical use of conventional cationic nanoparticles due to the non-specific cytotoxicity thereof, and in particular, they are not toxic to the human body and normal cells. Accordingly, the mixed-charge nanoparticles of the present invention may be advantageously used in medical and pharmaceutical applications such as prevention and treatment of solid cancer, blood cancer, and tumors.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

1. Mixed-charge nanoparticles comprising a nanocore to which a mixture of a positively charged ligand and a negatively charged ligand has been attached, and having a positive net surface charge.

2. The mixed-charge nanoparticles of claim 1, wherein a ratio (χ[+]:χ[−]) between the positively charged ligand and the negatively charged ligand on surfaces of the nanoparticles is 55:45 to 98:2.

3. The mixed-charge nanoparticles of claim 1, wherein the mixed-charge nanoparticles comprise a metal or metal oxide nanocore.

4. The mixed-charge nanoparticles of claim 3, wherein the nanocore is a gold, iron oxide, or silicon oxide nanocore.

5. The mixed-charge nanoparticles of claim 1, wherein the nanocore has a diameter of 4 nm to 12 nm.

6. The mixed-charge nanoparticles of claim 1, wherein the negatively charged ligand is any one or more selected from among the following Formulas (III) and (IV): 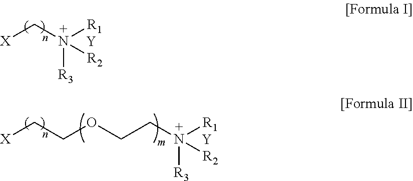
in Formula I or Formula II
X is a nanoparticle anchoring group selected from the group consisting of a thiol group, a carboxyl group, an amine group, a phosphine group, and a phenol-derived group;
Y is a halogen ion,
R₁, R₂ and R₃ are each independently selected from the group consisting of H and CH₃; and
n and m are each an integer of 1 or more.

7. The mixed-charge nanoparticles of claim 6, wherein the halogen ion is Cl⁻ or Br⁻.

8. The mixed-charge nanoparticles of claim 6, wherein X is a thiol group or a phenol-derived group,
wherein the thiol group is selected from the group consisting of 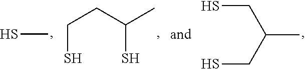
and
wherein the phenol-derived group is selected from the group consisting of

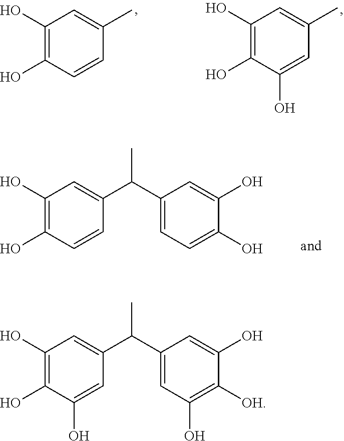

9. The mixed-charge nanoparticles of claim 1, wherein the negatively charged ligand is selected from among the following Formulas (III) and (IV): 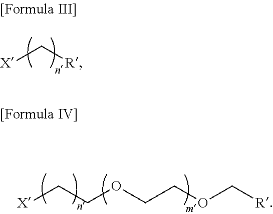
in Formula III and Formula IV
X′ is a nanoparticle anchoring group selected from the group consisting of a thiol group, a carboxyl group, an amine group, a phosphine group, and a phenol-derived group;
R′ is selected from the group consisting of a carboxyl group, sulphonates, and phosphonates; and
n′ and m′ are each an integer of 1 or more.

10. The mixed-charge nanoparticles of claim 9, wherein the X′ is a thiol group or a phenol-derived group, wherein the thiol group is selected from the group consisting of 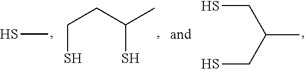
and the phenol-derived group is selected from the group consisting of

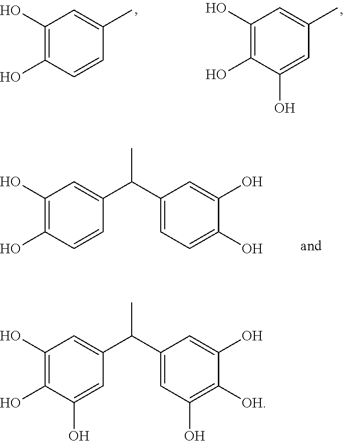

11. The mixed-charge nanoparticles of claim 9, wherein R′ is selected from the group consisting of

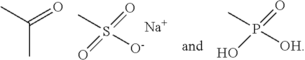

12. The mixed-charge nanoparticles of claim 1, having a zeta potential of 10 to 35 mV in water at pH 7.4.

13. The mixed-charge nanoparticles of claim 1, having a hydrodynamic diameter (DH) of 6 nm to 20 nm as determined by dynamic light scattering analysis.

14. The mixed-charge nanoparticles of claim 1, exhibiting cancer cell-specific cytotoxicity.

15. A composition for inducing cancer cell death containing the mixed charge nanoparticles of claim 1.

16. A pharmaceutical composition for preventing or treating cancer containing the mixed charge nanoparticles of claim 1.

17. The pharmaceutical composition of claim 16, wherein the cancer is solid cancer, metastatic cancer, or hematologic cancer.

18. (canceled)

19. (canceled)

20. A method for inducing cancer cell death comprising a step of treating a subject with the mixed charge nanoparticles of claim 1 or administering the mixed charge nanoparticles to a subject.

21. (canceled)

22. (canceled)

23. A method for preventing or treating cancer comprising a step of treating a subject with the mixed charge nanoparticles of claim 1 or administering the mixed charge nanoparticles to a subject.
